SEQUENCE LISTING (22 sequences)

SEQ ID NO: 1 - DNA, 47 bp, Artificial
  oligonucleotide
acactctttccctacacgacgctcttccgatctcctcaacataccgc

SEQ ID NO: 2 - DNA, 10 bp, Artificial
  oligonucleotide
tatgttgagg

SEQ ID NO: 3 - DNA, 42 bp, Artificial
  oligonucleotide
  misc_feature (11)..(20): n is a, c, g, or t
  misc_feature (42)..(42): n is a, c, g, or t
tgtccgaaagnnnnnnnnnnttttttttttttttttttttvn

SEQ ID NO: 4 - DNA, 14 bp, Artificial
  oligonucleotide
ctttcggacagcgg

SEQ ID NO: 5 - DNA, 10 bp, Artificial
  oligonucleotide
ctttcggaca

SEQ ID NO: 6 - DNA, 85 bp, Artificial
  oligonucleotide
  misc_feature (54)..(63): n is a, c, g, or t
  misc_feature (85)..(85): n is a, c, g, or t
acactctttccctacacgacgctcttccgatctcctcaacatatgtccgaaagnnnnnnnnnnttttttttttttttttttttvn

SEQ ID NO: 7 - DNA, 18 bp, Artificial
  oligonucleotide
aaatgtcccgcctgtctc

SEQ ID NO: 8 - DNA, 30 bp, Artificial
  oligonucleotide
taatacgactcactatagcgcgggacattt

SEQ ID NO: 9 - DNA, 28 bp, Artificial
  oligonucleotide
atttaggtgacactatagcgggacattt

SEQ ID NO: 10 - DNA, 10 bp, Artificial
  oligonucleotide
cgggacattt

SEQ ID NO: 11 - DNA, 85 bp, Artificial
  oligonucleotide
  misc_feature (54)..(63): n is a, c, g, or t
  misc_feature (85)..(85): n is a, c, g, or t
acactctttccctacacgacgctcttccgatctaaatgtcccggggttatagannnnnnnnnnttttttttttttttttttttvn

SEQ ID NO: 12 - DNA, 85 bp, Artificial; identical to SEQ ID NO: 6
  oligonucleotide
  misc_feature (54)..(63): n is a, c, g, or t
  misc_feature (85)..(85): n is a, c, g, or t

SEQ ID NO: 13 - DNA, 1029 bp, Homo sapiens
ctatcacctaagtgtgggctaatgtaacaaagagggatttcacctacatccattcagtcagtctttgggggtttaaagaaattccaaagagtcatcagaagaggaaaaatgaaggtaatgttttttcagacaggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccacaatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaa

SEQ ID NO: 14 - DNA, 58 bp, Artificial
  oligonucleotide
gtctcgtgggctcggagatgtgtataagagacagccacagaactgaaacatcttcagt

SEQ ID NO: 15 - DNA, 24 bp, Artificial
  oligonucleotide
ccctacacgacgctcttccgatct

SEQ ID NO: 16 - DNA, 25 bp, Artificial
  oligonucleotide
aatgatacggcgaccaccgagatct

SEQ ID NO: 17 - DNA, 30 bp, Artificial
  oligonucleotide
tcgccttagtctcgtgggctcggagatgtg

SEQ ID NO: 18 - DNA, 24 bp, Artificial
  oligonucleotide
ccctacacgacgctcttccgatct

SEQ ID NO: 19 - DNA, 20 bp, Artificial
  oligonucleotide
accatcctctatgagatcct

SEQ ID NO: 20 - DNA, 25 bp, Artificial
  oligonucleotide
gggtggagtcacatttctcagatcc

SEQ ID NO: 21 - DNA, 44 bp, Artificial
  oligonucleotide
caagcagaagacggcatacgagataccatcctctatgagatcct

SEQ ID NO: 22 - DNA, 50 bp, Artificial
  oligonucleotide
aatgatacggcgaccaccgagatctgggtggagtcacatttctcagatcc

(19) United States
(12) Patent Application Publication
(10) Pub. No.: US 2022/0403374 A1
(43) Pub. Date: Dec. 22, 2022

(54) OPTICALLY READABLE BARCODES AND SYSTEMS AND METHODS FOR CHARACTERIZING MOLECULAR INTERACTIONS

(71) Applicant: FLEXOMICS, LLC, Boston, MA (US)

(72) Inventor: Magali SOUMILLON, Boston, MA (US)

(21) Appl. No.: 17/639,819

(22) PCT Filed: Sep. 30, 2019
(86) PCT No.: PCT/US2020/049219

§ 371 Date: Mar. 2, 2022

Related U.S. Application Data
(60) Provisional application No. 62/895,159, filed on Sep. 3, 2019.

(51) Int. Cl. C12N 15/10; G01N 21/64

(52) U.S. Cl. CPC C12N 15/1065; G01N 21/6428; G01N 2021/6441

(57) ABSTRACT

A system and method are provided for simplifying and accelerating the screening and characterization of molecular interactions by high-throughput functional screening and sequencing of single cells. More specifically, a platform is provided which combines a solid support and an innovative method for capturing and barcoding of nucleic acids that allows simultaneous phenotyping and genotyping of >100, 000s of cells.

This application is a national stage application under 35 U.S.C. § 371 of International Application No. PCT/US2020/049219, filed Sep. 3, 2020, which claims the benefit of U.S. Provisional Patent Application Ser. No. 62/895,159, filed Sep. 3, 2019, which are hereby incorporated by reference in their entirety.

This invention was made with government support under Grant Number 1R43CA250673-01 awarded by the National Institutes of Health. The government has certain rights in the invention.

### FIELD

This application relates generally to optically readable barcodes and systems and methods for characterizing molecular interactions.

### BACKGROUND

Recent breakthroughs in understanding and exploiting the immune system's response to cancer has led to greatly improved management and treatment of many cancers. Development of molecular therapies such as immune checkpoint inhibitors and cellular therapies such as Chimeric Antigen Receptor (“CAR”) T-cells has transformed the prospects of some solid and liquid cancers, respectively. However, these novel treatments still suffer from lack of efficacy, toxicity, acquired resistance, and prohibitive costs. T-cell Receptor engineered T-cells (“TCR-T” cells) targeting tumor-specific antigens have emerged as a promising alternative to overcome some of these challenges. One barrier to the wide scale adoption of TCR-T therapies is the need to identify T-Cell receptor (“TCR”) sequences that recognize antigens expressed in specific cancers. Currently, there is no high-throughput approach to identify antigen-specific TCRs.

Small-scale methods have identified a number of antigen-specific TCR but matching antigen, TCR sequence, and T-cell response remains a challenging process. Assays characterizing T-cell responses, including cell proliferation, cytolytic activity, and cytokine expression have been traditionally applied to bulk populations and specific TCRs have been indirectly determined through the emergence of particular clones in response to the exposure to a particular antigen. While various methods relying on this bulk approach have identified TCR sequences targeting for example KRAS or TP53 mutations, they remain complex and time-consuming.

Alternatively, in silico-based approaches for neoantigen prediction are being developed to characterize cancer specific antigen in a more high-throughput manner. However, the complexity of both TCR structure and its interaction with a specific antigen makes modeling of the full complex extremely challenging. Furthermore, these predictions often return a large number of candidates from which only a handful effectively trigger antitumor responses in patients. Despite the high-frequency of mutations in cancer cells, immunogenic neoantigens are rare and high-throughput screening methods to validate neoepitopes and develop efficient targeted therapies based on these predictions are lacking.

Tumor heterogeneity, whether it is interpatient tumor heterogeneity or intratumor heterogeneity requires approaches that can identify thousands of functional TCR-antigen pairs for therapeutic use, some of which may be common to many tumors, others which may match only a single patient. In this context, throughput is a key element to increase the potential pool of patients that might be eligible for TCR-based therapies.

While Next-Generation Sequencing (NGS) has improved the accuracy and turnaround time to collect patients' tumor genotype information, obtaining the corresponding antigen-specific TCR sequence and assessing T-cell response remains challenging. Current technologies involve cumbersome APC/T-cell co-culture, labor-intensive serial limiting-dilution cloning, and indirect determination of TCR sequences through the time-consuming emergence of particular T-cell clones in bulk populations. Given the diversity of mutations present in cancers and the desirability of generating TCRs against multiple targets, these therapeutic companies would greatly benefit from accessing a technology that simplifies and expedites the identification of TCRs and their cognate neoantigens.

Another challenge is the identification of new antigen-specific antibodies. Whether it be for research, diagnostic, or therapeutic applications, for either polyclonal or monoclonal antibodies, and no matter the species, the identification of new antigen-specific antibodies currently rely on a few distinct approaches. The process generally starts with animal immunization and follows with identification and collection of antigen-specific B-cells for screening purposes.

In the case of the hybridoma based screening that was developed in the 1970s, antibody secreting cells are immortalized by fusion with an immortal myeloma cell line and serial limiting-dilution cloning is used to identify the cells secreting the antibody of interest. Although this method is extremely labor-intensive and time-consuming, it remains one of the most common approaches still used by leaders in the industry today due in large part to its relative low cost.

Alternatively, phage, yeast, or mammalian cell display approaches, developed in the 1980s, involve using a library of antibody fragments derived from cloning of the antigen-stimulated B cell mRNA repertoires. Although, this approach is a faster process that overcomes the throughput weakness of hybridomas, its difficulty, higher cost, and potential lower affinity results limits its broader adoption.

Finally, recent advances in microfluidics (arrays, droplets, gel-based), single-cell manipulation, and genomics have allowed the development of new promising technologies overcoming some of these limitations thanks to their ability to effectively culture non-immortalized antibody secreting cells. Unfortunately, the lack of functional data such as binding affinity and cross-species reactivity can result in large and costly validation efforts on a large number of candidates, making it difficult to implement as part of routine antibody discovery campaigns. None of these solutions are ideally suited for the difficult task of screening the massive immune diversity in the research of high-quality antibodies.

The present application is directed at overcoming these and other deficiencies in the art.

### SUMMARY

One aspect of the present application relates to an optically readable oligonucleotide probe comprising a plurality of nucleotide sequences. Each sequence of the plurality of nucleotide sequences comprises at least 3 distinct fluorescent labels comprising up to 7 unique fluorescent combinations to form a fluorescent word.

Another aspect of the present application relates to an oligonucleotide complex comprising a 5′ primer sequence and an optically readable well-identifying barcode comprising at least two words, where each word comprises a unique first plurality of nucleotide sequences, where the first plurality of nucleotide sequences are complementary to a second plurality of nucleotide sequences in an optically readable oligonucleotide probe, and where each nucleotide sequence of the second plurality of nucleotide sequences comprises at least 3 distinct fluorescent labels comprising up to 7 unique fluorescent combinations to form each of the fluorescent words. The oligonucleotide complex also includes a unique molecular identifier and a capture sequence.

A further aspect of the present application relates to an oligonucleotide-conjugated bead comprising a bead and a plurality of oligonucleotide complexes conjugated to the bead. Each oligonucleotide complex comprises a 5′ primer sequence; an optically readable well-identifying barcode comprising at least two words, where each word comprises a unique first plurality of nucleotide sequences, where the first plurality of nucleotide sequences are complementary to a second plurality of nucleotide sequences in an optically readable oligonucleotide probe, and where each nucleotide sequence of the second plurality of nucleotide sequences comprises at least 3 distinct fluorescent labels comprising up to 7 unique fluorescent combinations to form each of the fluorescent words; a plurality of unique molecular identifiers; and a plurality of capture sequences.

Yet another aspect of the present application relates to a solid support detection system comprising a solid support comprising a plurality of wells and a plurality of oligonucleotide-conjugated beads as described herein, where each of the plurality of oligonucleotide-conjugated beads comprises a unique optically readable well-identifying barcode, and where a plurality of the wells comprises one of the oligonucleotide-conjugated beads.

A further aspect of the present application is a method for preparing a solid support detection system for analyzing molecular interactions. This method involves providing a solid support comprising a plurality of wells, and depositing in a plurality of wells a single, unique, oligonucleotide-conjugated bead. The oligonucleotide-conjugated bead comprises oligonucleotides conjugated to the bead, where the oligonucleotides comprise a 5′ primer sequence, a well-identifying barcode sequence comprising at least two words, where each word comprises a unique first plurality of nucleotide sequences complementary to a probe. The oligonucleotide also comprises a unique molecular identifier and a 3′ capture sequence. The method further involves applying a series of flows, where each flow comprises a set of optically readable oligonucleotide probes. Each probe comprises a second plurality of nucleotide sequences complementary to the first plurality of nucleotide sequences, where each sequence of the second plurality of nucleotide sequences comprises at least 3 distinct fluorescent labels comprising up to 7 unique fluorescent combinations to form the fluorescent words.

Another aspect of the present application is directed to a high-throughput method of analyzing molecular interactions. This method involves providing the solid support detection system described herein; contacting the solid support with molecular components to be analyzed, where the molecular components are deposited in the plurality of wells; detecting phenotypic interactions between the molecular components; capturing RNA or DNA derived from the molecular components; transcribing captured RNA to DNA; enriching the DNA; sequencing the DNA; and matching sequences from the sequenced DNA to barcode sequences to identify the well location and, therefore, molecular interactions of interest among the molecular components.

The present application describes technology that allows the characterization and specific identification of millions of molecular interactions in a high-throughput manner. The screening and characterization of molecular interactions by high-throughput functional screening and downstream sequencing of target molecules of interest are accelerated and simplified. More specifically, described herein is a platform that combines a solid support and an innovative barcoding method for capturing and barcoding of nucleic acids that links well position identification on a solid support with phenotypic output and multiplex sequence output from 100,000s to millions of cells. The technology also allows rapid downstream cloning and use of target sequences from single identified cells.

The technology of the present application can be applied to the identification of TCR-antigen pairs, BCR-antigen pairs, phenotypic analysis of molecular interactions, drug candidate screening, characterization of circulating tumor cells, and numerous other applications. For example, while traditional enrichment methods have identified a number of TCR sequences that recognize common oncogenic neoantigens, high-throughput methods are now critical to greatly increase the number of targets against which TCRs with high affinity can be generated from dozens to thousands. The solid support based single cell platform of the present application is particularly suited for identifying and characterizing immunogenic neoantigens and their associated antigen-specific TCRs as one application.

Whereas droplet-based approaches are the current method of choice for large-scale single cell analysis including for tumor heterogeneity exploration, co-capturing and analyzing cell-to-cell interactions in such systems remains challenging. More specifically, co-capturing and monitoring cell to cell interaction in each individual droplet in a high-throughput manner is difficult. Similarly, culturing cells and adding or exchanging reagents required to perform functional assays on the captured cells in droplets has proven to be difficult.

In contrast, the unique physically partitioned plate of the present application allows one to directly interrogate antigen-T cell interaction as either TCR-bead or TCR-Antigen Presenting Cell (APC). And, unlike conventional microwell plate based methods, which are limited to the analysis of multiples of 96 or 384 cells and involve labor intensive plate manipulations, the high-density solid support of the present application, in conjunction with an innovative scalable barcoding system, enables the screening of >100,000s cells in a single experiment at very low per-cell costs (Table 1).

TABLE 1
Comparison of Antigen-Specific TCR Determination Strategies

| Assay | | | | |
|---|---|---|---|---|
| Performance | | | Droplet- | PRESENT |
| Features | Bulk approach | Plate-based | based | APPLICATION |
| Strategy | Entire bulk | Single-cell | Single-cell | Single-cell |
| | workflow | enrichment | enrichment | |
| | | from bulk | from bulk | |
| | | sample by | sample by | |
| | | FACS sorting | FACS sorting | |
| Antigen | APC or beads | Dextramers | Immudex | APC or beads |
| presentation | | | dCode | |
| | | | dextramers | |
| | | | pre- | |
| | | | enrichment | |
| Throughput | Requires clone | 96 to 384 | up to 10,000 | >100,000s cells |
| | emergence from | per plate | cells per | |
| | culture | | library and | |
| | | | 80,000 per | |
| | | | chip | |
| Functional | X | X | X | ✓ |
| Assay | | | | |
| Phenotyping | X | X | ✓ | ✓ |
| information | | | | |
| TCR- | Requires full | X | X | ✓ |
| sequence | synthesis, | | | |
| readily | | | | |
| available for | | | | |
| downstream | | | | |
| validation | | | | |

A key strength of the platform of the present application is the ability to directly link functional assays such as immunoassay readout and match this response to an antigen-specific TCR sequence. To do so, a groundbreaking method of the present application was developed for the capture and barcoding of nucleic acids to connect simultaneous phenotyping and genotyping at single cell resolution in a high-throughput manner. The method uses barcoded capture beads that can be optically decoded in the solid support allowing the fluorescent readout of an immunoassay to be spatially linked to the captured and sequenced TCR from the same cell.

A novel capture strategy of the present application is designed to barcode RNA derived from single T-cells encoding TCR alpha (α) and beta (β) chains which make up the TCR as well as transcripts from dozens of phenotypic markers including cytokines and transcription factors to further characterize activated T-cells. This unique targeted approach keeps sequencing costs minimal even at the massive >100,000s cells throughput. Additionally, to functionally test TCR sequences of interest a unique strategy was developed that uses a barcoding scheme to selectively enrich and extract the most promising full length TCR amplicons from a barcoded pool for direct downstream cloning and testing.

In addition to neoantigen-TCR screening, drug screening, and other applications, screening of gene-edited cells, screening of engineered T, B, or other cells, analysis and full characterization (RNA/DNA) of individual Circulating Tumor Cells (CTCs) enriched from blood, another application is B-Cell Receptor identification for antibody discovery/screening (Table 2). Due to the enrichment possibility, the technology described in the present application is a good tool to capture information from rare cells.

TABLE 2
Comparison of Antibody Discovery Strategies

| | | | Opto | | |
|---|---|---|---|---|---|
| Assay | | | Plasma B | | |
| Performance | | Surface | Discovery | Droplet | PRESENT |
| Features | Hybridoma | Display | Workflow | Based | APPLICATION |
| Strategy | Fusion of | Protein/peptide | Single cell | Droplet | High-throughput |
| | myeloma | display on | manipulation | based single | single cell |
| | and B cells | host surface | | cell capture | |
| | | | | and DNA- | |
| | | | | barcoded | |
| | | | | antigens | |
| Antigen | Plate Based | In-vitro | APC or | Immudex | APC or beads |
| Presentation | | surface | beads | | |
| | | display | | | |
| Throughput/ | Labor | 1e07-1e11 | ~10,000 | Up to | 100,000s of cells |
| Time to | intensive | | cells | 10,000 cells | |
| Answer | limiting | | automated | per library | |
| | dilution | | | and 80,000 | |
| | | | | per chip | |
| Time to | Months | Days | Days | Days | Days |
| Answer | | | | | |
| Functional | X | X | ✓ | X | ✓ |
| Assay | | | | | |
| Phenotyping | X | X | X | ✓ | ✓ |
| Information | | | | | |
| Ig-sequence | Hc and Lc | Ig sequence | X | X | ✓ |
| Readily | genes can be | available for | | | |
| Available for | cloned | downstream | | | |
| Downstream | | validation in | | | |
| Validation | | some format | | | |
| Cost | $ | $$ | $$$ | $$ | $ |

The molecular structures, systems, and methods described herein allow the characterization and identification of hundreds of thousands (or more) of molecular interactions in a high-throughput manner. Such throughput is crucial to allow the development of safe and efficient personalized therapies. For example, given the multiplicity of mutations present in cancers, accessing and compiling unique information pertaining to the cancer specific antigen recognition and immunogenicity at large scale and in a timely manner would be a game changer for the design of such therapies.

### BRIEF DESCRIPTION OF THE DRAWINGS

The preceding summary, as well as the following detailed description of the disclosed system and method, will be better understood when read in conjunction with the attached drawings. It should be understood, however, that neither the system nor the method is limited to the precise arrangements and instrumentalities shown.

FIGS. 1A-D are schematic illustrations showing the distinction between conventional (prior art) fluorescent labeling and barcode word detection (FIGS. 1A-B) and one embodiment of the optically readable oligonucleotide probes of the present application (FIGS. 1C-D).

FIGS. 2A-B are schematic illustrations of an embodiment of a method of generating fluorescent probes according to the present application. FIG. 2A illustrates the use of T7 polymerase to transcribe a DNA template to generate RNA probes with incorporated fluorescently labeled nucleotides. FIG. 2B illustrates a similar process where the DNA template is attached to beads.

FIGS. 3A-B are schematic illustrations of one embodiment of an oligonucleotide complex of the present application. FIG. 3A shows the general oligonucleotide structure of one embodiment of a well-identifying barcode of the present application and FIG. 3B shows the formation of an oligonucleotide complex as described herein achieved with a series of six separate flows.

FIG. 4 is a schematic illustration of one embodiment of an oligonucleotide-conjugated bead of the present application containing a universal primer; a well-identifying barcode containing 6 words, where the well-identifying barcode is identical for all oligonucleotides on one bead; a unique molecular identifier different for different oligonucleotides on one bead; and a capture sequence, which can be different for different oligonucleotides on one bead.

FIGS. 5A-C are photographic images showing successful experimental results of bead and cell capture in solid support plates according to an embodiment of the present application. FIG. 5A shows the solid support separated into 16 distinct sections with a gasket and loading of the solid support. FIG. 5B shows beads loaded on the solid support. FIG. 5C shows co-loading of beads (blue) and MCF7 cells (red).

FIG. 6 is a photograph showing the use of optic fiber features to homogenize fluorescent detection.

FIG. 7 is a schematic illustration showing a method of single cell response measurement for a high number of cells and a high number of chemical compounds (drugs) in parallel (using a gasket positioned on the solid support) at single cell resolution in accordance with an embodiment of the methods of the present application.

FIGS. 8A-C are schematic illustrations showing different types of capture primers that can be carried and potentially delivered by oligonucleotide-conjugated beads as described herein, in accordance with the methods of the present application. FIG. 8A shows capture of mRNA and amplification of target genes. FIG. 8B shows amplification of TCR sequences. FIG. 8C shows amplification of target DNA sequences after photocleavage and release of oligonucleotides.

FIGS. 9A-B shows the successful demonstration of the feasibility of a targeted version of a single cell RNA barcoding and sequencing (SCRB-seq) for capture of gene expression of key marker genes in accordance with an embodiment of a method of the present application. FIG. 9A shows an electropherogram with the products of four distinct genes ranging from 400 bp to 1000 bp. FIG. 9B is a schematic illustration showing the capture of mRNA by the capture sequence and amplification of genes of interest using gene-specific primers.

FIGS. 10A-H show the validation of TCR-seq library preparation in comparison with commercially available TCR-seq methods. FIGS. 10A-B are a schematic illustration of a T-Cell Receptor and B-Cell Receptor sequence enrichment process from barcoded cDNA. In FIG. 10A, the oligo-dT capture sequence anneals to mRNA. In FIG. 10B, targeted enrichment of the TCR or BCR sequence is performed with nested PCR to capture TCR and BCR JDV sequences. FIG. 10C shows the length (by base) of the CDR3 sequence. FIG. 10D shows and the frequency of J genes and FIG. 10E shows the frequency of V genes in a TCR-seq experiment of the present application. FIGS. 10F-H show CDR3 length, J gene frequencies and V gene frequencies using an Adaptive Biotechnologies method.

FIGS. 11A-C are a schematic illustration showing a process of re-enrichment of transcript/sequence from specific cells from pooled cDNA and/or DNA by using the well-identifying barcode position information and sequence to amplify sequences from a chosen cell in accordance with methods of the present application. FIG. 11A shows a unique well-identifying barcode associated with TCR sequences from different cells. FIG. 11B shows the pool of TCR sequences from all cells of the solid support. FIG. 11C shows selective re-enrichment for specific TCR sequences using nested primers corresponding to the specific barcode for that cell and well for use in later applications.

FIGS. 12A-B illustrate a method of the present application for drug screening application. FIG. 12A shows a schematic illustration of a phenotypic assay of a cell in a well of a solid support measured by a fluorescent antibody. FIG. 12B shows an illustration of different areas of a solid support being treated with different chemical compounds in accordance with methods of the present application.

FIG. 13 is a schematic illustration showing an embodiment of a method of the present application for circulating tumor cell analysis.

FIGS. 14A-D are a schematic illustration showing a workflow diagram illustrating an embodiment of a method of phenotypic and genotypic analysis of the present application with T-cells and APC cells. Interaction of T-Cell and APC cell in the well leading to production of compounds detectable by fluorescent assays and well position are followed by detection of target sequences that are barcoded with the well-identifying barcode. Both TCR-sequences and other sequences of interest can be captured. A specific amplicon (e.g. a T-cell receptor from a specific cell from the barcoded pooled cDNA) can be further enriched for downstream cloning and functional validation.

FIGS. 15A-M are schematic illustrations showing a workflow diagram illustrating an embodiment of a method for the characterization and specific identification of hundreds of thousands of molecular interactions in a high-throughput manner of the present application. FIG. 15A shows a solid support according to one embodiment of the present application in a picowell plate formation. FIG. 15B shows loading of the optically barcoded beads into the solid support (and positioned into wells). FIG. 15C shows the sequential flow of fluorescently labeled probes over the solid support in which the probes anneal to their complementary sequences of the oligonucleotide barcoded words conjugated to the bead. Fluorescent signals are captured after each flow. FIG. 15D shows an example output of the fluorescent signal for a well indicating the presence (1) or absence (0) of a particular fluorescent label for each flow. The known sequence for each probe and label combination and output signal for each probe are used to decode a well-identifying barcode in each well, allowing the integration of the barcode sequence with the output of phenotypic assays. FIG. 15E shows the loading of cells into the mapped optically barcoded bead solid support. FIG. 15F shows the loading of additional cells, antigen coated beads, or other molecule of interest into the wells. FIG. 15G shows the application of a phenotypic assay such as an immunofluorescence assay revealing productive interactions between molecules by position in the solid support. FIG. 15H shows the lysing of cells to release cellular contents. FIG. 15I shows hybridization of mRNA or other sequences to capture sequences on the oligonucleotide-conjugated bead. FIG. 15J shows the application of reverse transcriptase, gene specific primers, etc. to amplify target sequences of interest. FIG. 15K shows the sequencing of target sequences of interest that include the mapped barcode sequences. FIG. 15L shows the integration of the phenotypic data by well position with the optically mapped well-identifying barcode sequence. FIG. 15M shows re-enrichment of selected sequences based on phenotypic and genotypic data.

FIGS. 16A-C are an illustration and photographic images of an oligonucleotide-conjugated bead with two words complexed with two fluorescent word probes. FIG. 16A is an illustration of the oligonucleotide-conjugated bead complexed to two fluorescent words. FIG. 16B shows photographic images of fluorescence imaged in two different channels to capture the fluorescence from an Alexa488 labeled probe complex (FITC channel) and a Cy3 labeled probe complex (Cy3 channel). The oligonucleotide-conjugated bead in FIG. 16B was constructed through ligation of Word 1 and Word 2 on separate oligonucleotides. FIG. 16C shows photographic images of the same probes imaged in two channels (FITC channel and Cy3 channel) in a complex with an oligonucleotide-conjugated bead that was synthesized.

FIGS. 17A-B show a schematic illustration and a photographic image showing loading of different sections of a solid support with different beads labeled with green or red fluorescent labels.

FIGS. 18A-B are photographic images of an oligonucleotide-conjugated bead with a barcode word visualized by hybridizing the oligonucleotide-conjugated bead with the generated internal fluorescently labelled nucleotide, complementary probes generated by a T7 polymerase starting with a single stranded template (bright field, FIG. 18A and FITC fluorescent channel, FIG. 18B).

FIGS. 19A-B are photographic images of an oligonucleotide-conjugated bead with a barcode word visualized by hybridizing the oligonucleotide-conjugated bead with the generated internal fluorescently labelled nucleotide, complementary probes generated by a T7 polymerase starting with a double-stranded template (bright field, FIG. 19A and FITC fluorescent channel, FIG. 19B).

FIGS. 20A-B are photographic images of oligonucleotide-conjugated beads with barcode words hybridized with the probe of FIGS. 19A-B, but imaged in different fluorescent channels (Cy3, FIG. 20A and DAPI, FIG. 20B).

FIGS. 21A-B are photographic images of oligonucleotide-conjugated beads with a barcode word visualized by hybridizing the oligonucleotide-conjugated bead with the generated internal fluorescently labelled nucleotide, complementary probes generated by a SP6 polymerase starting with a double-stranded template (bright field, FIG. 21A and FITC fluorescent channel, FIG. 21B).

FIGS. 22A-C are photographic images of oligonucleotide-conjugated beads with a barcode word visualized by hybridizing the oligonucleotide-conjugated bead with 5′ and 3′ fluorescently labelled nucleotides, complementary probes (bright field, FIG. 22A, FITC fluorescent channel, FIG. 22B, and Cy3 fluorescent channel, FIG. 22C).

FIGS. 23A-C are photographic images of two types of oligonucleotide-conjugated beads (having either barcode word 1 or barcode word 2) loaded onto a solid support (FIG. 23A) and probe complexes with the oligonucleotide-conjugated beads corresponding to a probe to word 1 labeled with FITC (FIG. 23B), or to a probe to word 2 labeled with Cy3 (FIG. 23C).

FIGS. 24A-B are photographic images of the solid support loaded with Jurkat cells stained with CellTrace CSFE and imaged in bright field (FIG. 24A) or the FITC fluorescent channel (FIG. 24B).

FIGS. 25A-B are photographic images of a solid support after flows of two types of oligonucleotide-conjugated beads with either barcode words 1 and 2 or words 3 and 4 and complexed with a probe to word 1 were loaded, in addition to flows of Jurkat cells (T-Cells) stained green, and KG-1 cells stained blue. All beads were detected and mapped in bright field (lower right corner of FIG. 25A). Only oligonucleotide-conjugated beads with word 1 were detected in the RFP channel (upper right corner of FIG. 25A). Oligonucleotide-conjugated beads with word 2 were not detected in the RFP channel (compare upper right corner of FIG. 25A with lower right corner of FIG. 25B). KG-1 cells were detected in the DAPI channel (lower left corner of FIG. 25A) and Jurkat cells emitted fluorescent signal in the GFP channel (upper right corner of FIG. 25A). FIG. 25B is a merged overlay of the four channels.

FIGS. 26A-B are photographic images showing loading of cells stained with RNASelect Green Fluorescent stain before (FIG. 26A) and after flowing lysis buffer (FIG. 26B).

FIGS. 27A-D are photographic images showing images of the solid support after two types of photo-cleavable oligonucleotide-conjugated beads complexed with probes to two different barcode words were loaded. FIG. 27A shows oligonucleotide-conjugated beads complexed with a probe to barcode word 3 (FITC channel), and FIG. 27B shows oligonucleotide-conjugated beads complexed with a probe to barcode word 1 (Cy3 channel). FIGS. 27 C-D show the release of the oligonucleotides from the beads after UV treatment.

FIGS. 28A-B show a schematic illustration and a photographic image of gene specific amplification using a capture sequence and a gene specific primer for interleukin-2 (IL-2). FIG. 28A illustrates the capture of mRNA with the capture sequence on the oligonucleotide-conjugated beads followed by targeted RNA-seq enrichment. FIG. 28B shows amplification of IL-2 using this process.

### DETAILED DESCRIPTION

A first aspect of the present application relates to an optically readable oligonucleotide probe comprising a plurality of nucleotide sequences, where each sequence of the plurality of nucleotide sequences comprises at least 3 distinct fluorescent labels comprising up to 7 unique fluorescent combinations to form a fluorescent word. The optically readable oligonucleotide probes described herein enable the formation of oligonucleotides complexes, with oligonucleotide-conjugated beads, which are used with a solid support detection system to carry out high-throughput methods of analyzing molecular interactions, all of which is described in further detail infra.

Optically readable oligonucleotide probes of the present application comprise a plurality of nucleotide sequences, each of which is generally between 6-20 nucleotides in length, although other lengths may also be used. In certain embodiments, the nucleotide sequences that form the plurality of nucleotide sequences each comprise 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, or 20 nucleotides in length.

These relatively short nucleotide sequences can be designed with computer assistance to optimize sequence composition to match target hybridization parameters and avoid cross hybridization to complementary well-identifying barcodes (discussed infra).

Each of the nucleotide sequences in the plurality of nucleotide sequences in the optically readable probe provides a substrate or structural backbone to which fluorescent labels may be attached. Each nucleotide sequence of the plurality of nucleotide sequences of the probe comprises at least 3 distinct fluorescent labels comprising up to 7 unique fluorescent combinations to form a single fluorescent “word.” The fluorescent “words” may then be “read” or imaged by standard methods of fluorescent imaging. When formed into oligonucleotide complexes through complementary nucleotide hybridization to oligonucleotide-conjugated beads, these combinations of “words” become identifiers for locations (e.g., well locations) on a solid support detection system to identify cellular interactions or specific cellular components of interest via high-throughput methods of analyzing molecular interactions.

In an optically readable oligonucleotide probe, the plurality of nucleotide sequences that make the probe are each different known sequences from each other.

Thus, each fluorescent “word” has fluorescently labeled nucleotides that are capable of hybridizing to a plurality of complementary sequences of an unlabeled barcode (discussed infra) to form a complex and emit a fluorescently detectable signal that can be “read” by standard fluorescence imaging.

Fluorescent labels may be attached to the nucleotide sequences of the probe at any location in the nucleotide sequence, including at the 5′ end of the nucleotide sequence, at the 3′ end of the nucleotide sequence, at internal nucleotide positions, or any combination thereof.

The plurality of nucleotide sequences comprise at least 3 distinct fluorescent labels comprising up to 7 unique fluorescent combinations to form a fluorescent word. Higher numbers of distinct fluorescent labels may also be used, which creates a higher number of possible unique fluorescent combinations to form a higher number of unique fluorescent words. For example, the plurality of nucleotide sequences may each comprise at least 4 distinct fluorescent labels, in which case the optically readable oligonucleotide probe could have up to 15 unique fluorescent combinations to form a fluorescent word. In another example, the plurality of nucleotide sequences each comprises at least 5 distinct fluorescent labels, in which case the optically readable oligonucleotide probe could have up to 31 unique fluorescent combinations to form a fluorescent word. In yet another example, the plurality of nucleotide sequences may each comprise at least 6 distinct fluorescent labels, in which case the optically readable oligonucleotide probe could have up to 63 unique fluorescent combinations to form a fluorescent word. In still another example, the plurality of nucleotide sequences each comprise at least 7 distinct fluorescent labels, in which case the optically readable oligonucleotide probe could have up to 127 unique fluorescent combinations to form a fluorescent word. Higher numbers of distinct fluorescent labels could also be used in the probes described herein, which creates even higher numbers of possible unique fluorescent combinations.

Fluorescent labels that may be used in conjunction with the nucleotide sequences of the optically readable oligonucleotide probes are known in the art, and may include fluorescent deoxyribonucleoside analogs. In some embodiments, the fluorescent labels are fluorescent ribonucleoside analogs. In some embodiments, the fluorescent labels are fluorescent dideoxy analogs.

Suitable fluorescent labels for use with the optically readable oligonucleotide probes include, without limitation, Alexa405, Alexa488, Alexa456, Alexa605, FAM, HEX, Cy3, Fluorescein-12-GTP, Cyanin-5-CTP, Cyanin-3-CTP, Cyanin-3-UTP, Fluorescein-12-ATP, Texas red-dATP, Cyanine-3-dCTP, cyanine-5-dGTP, or Fluorescein-12-dUTP., Fluorescein-12-dUTP, Coumarin-5-dUTP, Tetramethylrhodamine-6-dUTP, Texas Red®-5-dUTP, Napthofluorescein-5-dUTP, Fluorescein Chlorotriazinyl-4-dUTP, Pyrene-8-dUTP, Diethylaminocoumarin-5-dUTP, Cyanine 3-dUTP, Cyanine 5-dUTP, Coumarin-5-dCTP, Fluorescein-12-dCTP, Tetramethylrhodamine-6-dCTP, Texas Red®-5-dCTP, Lissamine-5-dCTP, Napthofluorescein-5-dCTP, Fluorescein Chlorotriazinyl-4-dCTP, Pyrene-8-dCTP, Diethylaminocoumarin-5-dCTP, Cyanine 3-dCTP, Cyanine 5-dCTP, Coumarin-5-dATP, Diethylaminocoumarin-5-dATP, Fluorescein-12-dATP, Fluorescein Chlorotriazinyl-4-dATP, Lissamin-5-dATP, Napthofluorescein-5-dATP, Pyrene-8-dATP, Tetramethylrhodamine-6-dATP, Texas Red®-5-dATP, Cyanine 3-dATP, Cyanine 5-dATP, Coumarin-5-dGTP, Fluorescein-12-dGTP, Tetramethylrhodamine-6-dGTP, Texas Red®-5-dGTP, Lissamin-5-dGTP, Fluorescein-12-UTP, Coumarin-5-UTP, Tetramethylrhodamine-6-UTP, Texas Red®-5-UTP, Lissamine-5-UTP, Napthofluorescein-5-UTP, Fluorescein Chlorotriazinyl-4-UTP, Pyrene-8-UTP, Cyanine 3-UTP, Cyanine 5-UTP, Coumarin-5-CTP, Fluorescein-12-CTP, Tetramethylrhodamine-6-CTP, Texas Red®-S-CTP. Lissamine-S-CTP, Napthofluorescein-S-CTP, Fluorescein Chlorotriazinyl-4-CTP, Pyrene-8-CTP, Cyanine 3-CTP, Cyanine 5-CTP, Coumarin-5-ATP, Fluorescein-12-ATP, Tetramethylrhodamine-6-ATP, Texas Red®-5-ATP, Lissamine™-5-ATP, Coumarin-5-GTP, Fluorescein-12-GTP, Tetramethylrhodamine-6-GTP, Texas Red®-5-GTP, Lissamine-5-GTP, Fluorescein-12-ddUTP, FAM-ddUTP, ROX-ddUTP, R6G-ddUTP, TAMRA-ddUTP, JOE-ddUTP, R110-ddUTP, Fluorescein-12-ddCTP, FAM-ddCTP, ROX-ddCTP, R6G-ddCTP, TAMRA-ddCTP, JOE-ddCTP, R110-ddCTP, Fluorescein-12-ddGTP, FAM-ddGTP, ROX-ddGTP, R6G-ddGTP, TAMRA-ddGTP, JOE-ddGTP, R110-ddGTP, Fluorescein-12-ddATP, FAM-ddATP, ROX-ddATP, R6G-ddATP, TAMRA-ddATP, JOE-ddATP, and R110-ddATP. Other fluorescent labels may also be used.

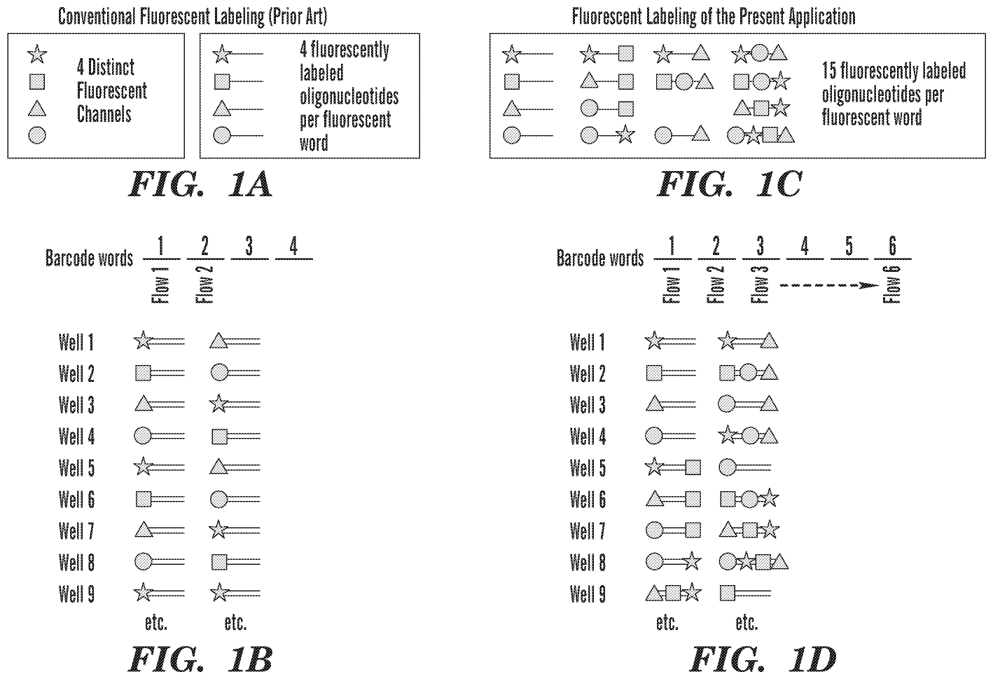

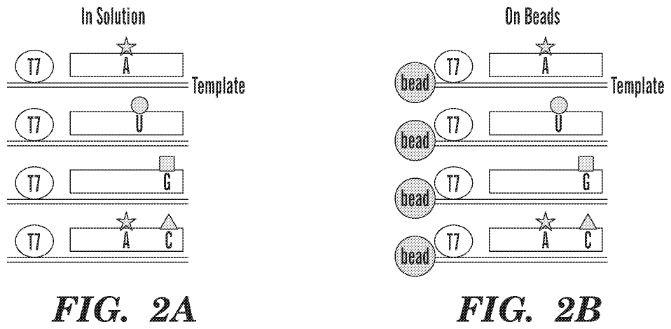

Turning now to FIGS. 1A-D, fluorescent labeling of nucleotide sequences to form optically readable oligonucleotide probes comprising “words” is shown. Specifically, FIGS. 1A and 1C compare (i) conventional (prior art) fluorescently labeled probes where only one fluorescent label (represented by one of the four unique shapes—star, square, triangle, and circle) is assigned to each nucleotide sequence of the probe (FIG. 1A) to (ii) the technology described in the present application in which different combinations of fluorescent labels may also be assigned to different nucleotide sequences of the probe to create a greater number of distinct nucleotide sequences of an optically readable probe using the same number (four) of distinct fluorescent labels (FIG. 1C). As illustrated in FIG. 1C, each fluorescently labeled nucleotide sequence of a probe comprises one, two, three, or all four of the fluorescent labels to create a total of 15 fluorescent combinations for each labeled nucleotide sequence in accordance with an embodiment of the present application.

FIGS. 1B and 1D show the hybridization of optically readable oligonucleotide probes comprising a plurality of fluorescently labeled nucleotide sequences as illustrated in FIGS. 1A and 1B to the complementary sequences of unlabeled barcode sequences to form barcode “words”. In the prior art (FIG. 1B), each barcode word is formed by only four combinations of fluorescently labeled nucleotide sequences of a probe, each having a single fluorescent label. Hybridization of each probe to complementary barcode words (described infra) occurs through a “flow” in which a solution containing the probes is flooded over the unlabeled barcodes and nucleotide sequences in the probe having complementary sequences in the barcodes hybridize.

In the example shown in FIG. 1B, the first barcode word (represented by horizontal lines) is detected in Flow 1 by hybridizing the nucleotide sequences of the first optically readable oligonucleotide probe to the barcodes and detecting the fluorescence. Each fluorescent label can be detected in different “channels” or fluorescent wavelengths. The second barcode is detected in Flow 2 with the second optically readable oligonucleotide probe. The prior art combination of probes and signals from each barcode word only allows the unique identification of 4² (16) different sequences for two barcode words, or 4⁴ (256) unique sequences for four barcode words, or up to 4⁶ (4096) unique sequences for six barcode words.

In contrast, FIG. 1D shows the hybridization of fluorescently labeled probes to unlabeled barcodes to form barcode words according to the present application. In the illustrated embodiment of the present application shown in FIG. 1D, each flow can contain up to 15 different sequences per optically readable oligonucleotide probe to form a fluorescent word, which allows higher multiplexing and speeds-up the decoding process with fewer rounds of hybridizations required. The increased complexity of probe molecules combined with six barcode words allows the unique identification of up to 15⁶ (11,390,625) unique labels (for example). (As discussed infra, each unique label can be used to identify the contents of a single well of a solid-support array.) The fluorescent output from each flow of an optically readable oligonucleotide probe comprising a plurality of nucleotide sequences is imaged upon hybridization with its complementary sequences of the barcode word. This is then followed by a flow of another optically readable oligonucleotide probe comprising a plurality of nucleotide sequences, which hybridizes with the second unlabeled barcode word and is imaged, and so on. This process is described in further detail infra.

Alternatively, the position of a given word may be detected using a single dedicated channel approach. In this method, the maximum number of barcode combinations is determined by the number of flows of detection probes. For example, in one flow there would be one each of a blue, red, green, and yellow labeled probe of known sequences, each hybridizing to a different barcode word. As a non-limiting example, in the first flow, the blue labeled probe would hybridize to the complementary sequences in word 1, the red labeled probe would hybridize to the complementary sequences in word 2, the green labeled probe would hybridize to the complementary sequences in word 3 and the yellow labeled probe would hybridize to the complementary sequences in word 4. In a second flow, one probe of each color corresponding to a second group of sequences would each hybridize to their complementary sequences in words 1-4 as before (i.e. blue for word 1, red for word 2, etc.). Over 10 flows, there would be 10 blue probes to detect word 1, 10 red probes to detect word 2, 10 green probes to detect word 3, and 10 yellow probes to detect word 4. In this case the total number of possible combinations of unique barcode sequences will be 10×10×10×10=10,000 distinct combinations. The advantage of this approach is that there is no need to eliminate the fluorescence from the previous flow as a well containing the barcode will only be positive for one specific color in one given flow.

The optically readable oligonucleotide probes allowing the identification of each “word” of the present application can be synthesized as DNA or RNA oligonucleotides and labelled on 5′, 3′, or internal positions with fluorophores such as Alexa405, Alexa488, Alexa456, Alexa605, FAM, HEX, and Cy3. Fluorescent DNA probes can be generated by integrating fluorescent dNTPs such as Texas red-dATP, Cyanine-3-dCTP, cyanine-5-dGTP, or Fluorescein-12-dUTP, as examples. DNA probes can be generated through 5′ end labeling, labeling by PCR, 3′ end labeling, single nucleotide terminator labeling, random priming, nick translation or reverse transcription, as non-limiting examples. Alternatively, the complementary fluorescent probes can be generated as RNA probes via in vitro transcription of barcode template using T7 promoters and can either integrate fluorescent NTPs such as Fluorescein-12-GTP, Cyanin-5-CTP, Cyanin-3-CTP, Cyanin-3-UTP, Fluorescein-12-ATP and/or be labeled with fluorophores.

FIGS. 2A-B show exemplary illustrations of embodiments of a method of generating optically readable oligonucleotide RNA probes. In FIG. 2A, DNA templates comprising a T7 promoter in solution (FIG. 2A) are shown, and in FIG. 2B, DNA templates comprising a T7 promoter attached to beads are shown. A mixture of nucleotides and T7 polymerase is added to generate RNA probes (rectangles) with all the different combination of sequences required for the barcode word decoding. The pool of nucleotides used during the in vitro transcription step comprises a mix of labeled and non-labeled NTPs specific to the labeling that needs to be achieved, from 1 to up to the maximum number of fluorescent labels used for each given probe. In this exemplary method, any suitable polymerase may be used to generate the probes. In some embodiments, the probe is generated using a single subunit DNA-dependent RNA-polymerase. In other embodiments, the RNA-polymerase is any one of T7, SP6, or T3 polymerases.

A kit such as mirVana miRNA probe construction kit can be used for probe generation (ThermoFisher Scientific, Waltham, Mass.). Excess leader or other sequence in the probes can be removed by annealing a reverse complementary sequence and using RNAse H digestion to remove the double stranded sequence. In some embodiments, generating the probes includes the incorporation of uracil to allow degradation of the fluorescent probes by USER (Uracil-Specific Excision Reagent) enzyme to allow for fluorescent signal removal.

In the discussion that will now follow, oligonucleotide complexes are described, which include the optically readable oligonucleotide probes hybridized to complementary nucleotide sequences in well-identifying barcodes. These well-identifying barcodes become optically readable when hybridized to complementary nucleotide sequences in the optically readable oligonucleotide probes.

Thus, another aspect of the present application relates to an oligonucleotide complex comprising a 5′ primer sequence; an optically readable well-identifying barcode comprising at least two words, where each word comprises a unique first plurality of nucleotide sequences, where the first plurality of nucleotide sequences are complementary to a second plurality of nucleotide sequences in an optically readable oligonucleotide probe, and where each nucleotide sequence of the second plurality of nucleotide sequences comprises at least 3 distinct fluorescent labels comprising up to 7 unique fluorescent combinations to form each of the fluorescent words; a plurality of unique molecular identifiers; and a plurality of capture sequences.

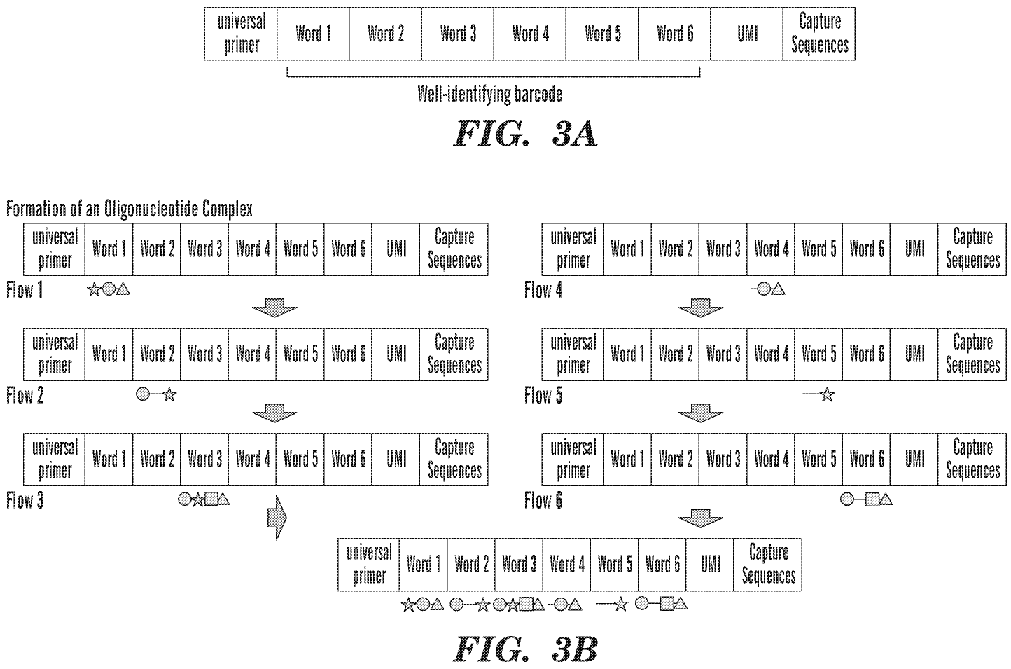

FIGS. 3A-B illustration one embodiment of the oligonucleotide complex of the present application. One element of the oligonucleotide complex includes the oligonucleotide illustrated in FIG. 3A, which includes a common 5′ primer sequence (“universal primer”) for use in downstream applications. The 5′ primer sequence generally comprises 6-50 nucleotides in length. The oligonucleotide complex further comprises a well-identifying barcode, which is labeled in FIG. 3A (“Well-identifying barcode”), and is shown to be formed of 6 words (“Word 1”, “Word 2”, “Word 3”, “Word 4”, “Word 5”, and “Word 6”), where each word comprises a unique first plurality of nucleotide sequences. As used herein, a “well-identifying barcode” means a nucleic-acid based barcode sequence used to identify a specific well in a solid support that can be detected both by fluorescence and by sequencing. As discussed in more detail infra, the “well” referred to in the “well-identifying barcode” is a well, or reaction chamber on a solid support, which typically includes a bead comprising a plurality of oligonucleotide complexes conjugated to the bead. Well-specific phenotypic information and sequencing information related to the molecular components in a single well on a solid support are linked through the well-identifying barcode as described in detail infra. Each well-identifying barcode is designed as a succession of “words”. In some embodiments, a well-identifying barcode comprises at least two words. In some embodiments, a well-identifying barcode comprises at least 3 words, at least 4 words, at least 5 words, at least 6 words, at least 7 words, at least 8 words, at least 9 words, at least 10 words, or more than 10 words. In some embodiments, a word is comprised of 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, or more than 20 nucleotides. Exemplary oligonucleotide complexes are shown in FIG. 3B in which each fluorescent word hybridizes to a complementary word in the well-identifying barcode in a successive manner as indicated by Flows 1-6. Hybridization of complementary sequences in the (i) well-identifying barcode and the (ii) optically readable oligonucleotide probe include DNA/DNA hybrids as well as RNA/DNA hybrids, RNA/RNA hybrids, and synthetic nucleotide hybrids.

It is desirable for the nucleotide sequences in the well identifying barcode to hybridize only to complementary nucleotide sequences in the optically readable oligonucleotide probe. To prevent non-specific annealing, each fluorescent word and its complementary word in the well-identifying barcode may be designed with a distance equal to at least half of its length, i.e., considering all nucleotides and their individual position in a word, two words are designed as different for at least 50% of their sequence. Homopolymers, i.e., stretches of the same nucleotides are excluded. To prevent unwanted annealing of the nucleotide sequences in the optically readable oligonucleotide probes, the nucleotide sequences in generated well-identifying barcode words are screened for homologies with genomic, transcriptomic, and universal synthetic sequences used for general amplification and sequencing, and those sequences are eliminated from use as a word.

The oligonucleotide complex also comprises a unique molecular identifier (“UMI”). The UMI comprises random sequences to uniquely tag a sequence arising from RNA or DNA to allow discrimination in downstream sequencing analysis between reads arising from individual molecules and those arising from PCR amplification. See, e.g., Islam et al., “Quantitative Single-Cell RNA-seq with Unique Molecular Identifiers,” Nature Methods 11:163-166 (2014), which is hereby incorporated by reference in its entirety.

The oligonucleotide complex also comprises a capture sequence. As used herein, a “capture sequence” means an oligonucleotide sequence that can be used to capture a sequence of interest for amplification and sequencing. Exemplary types of capture sequences include oligo-dT for mRNA capture, gene specific primer sequences for reverse TCR and B-Cell Receptor (“BCR”) sequencing, and PCR primers for DNA enrichment and mutation detection. Non-limiting examples of the capture sequence include an oligo-dT sequence, a TCR sequence, a BCR sequence, a GATA3 sequence, a TBET sequence, FOXP3 sequence, RORC sequence, RUNX1 sequence, RUNX3 sequence, BCL6 sequence, IL2 sequence, IL10 sequence, IL12A sequence, IL13 sequence, IL17A sequence, IFNG sequence, TNFA sequence, TGFB sequence, PRF1 sequence, a GZMB sequence, and combinations thereof.

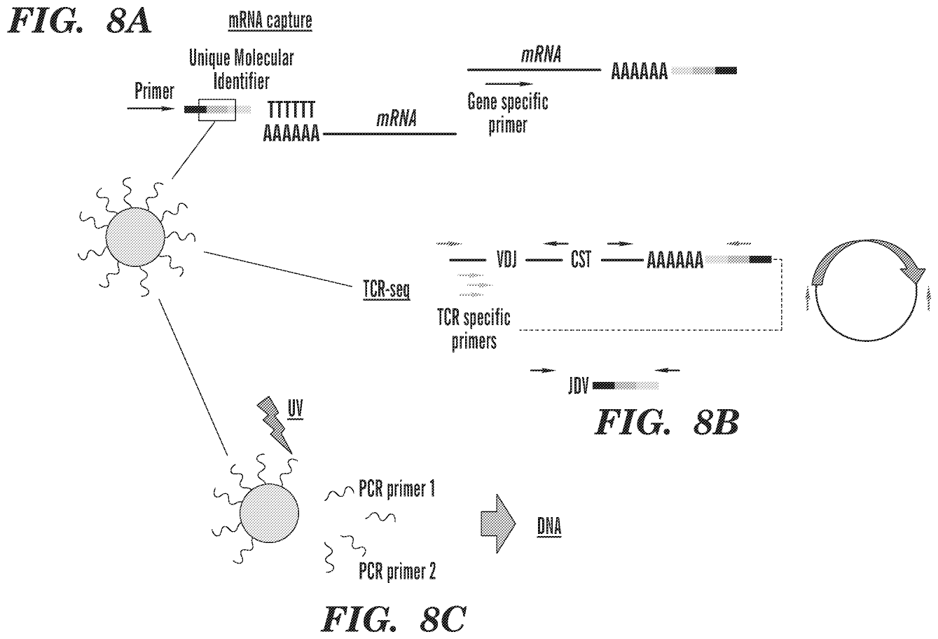

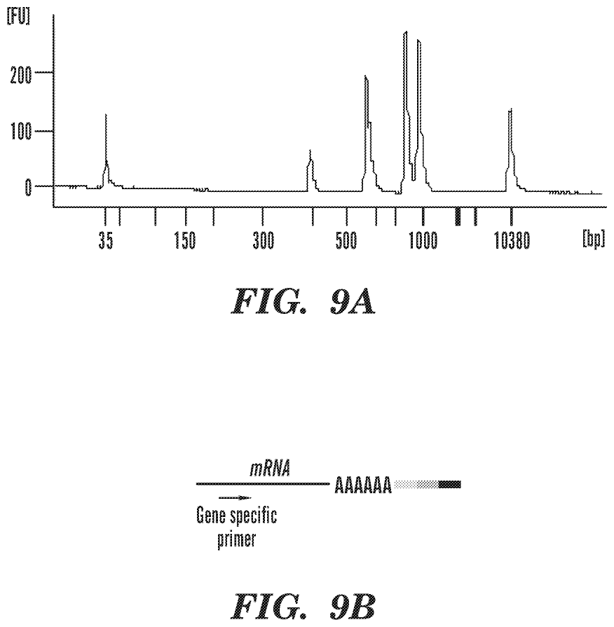

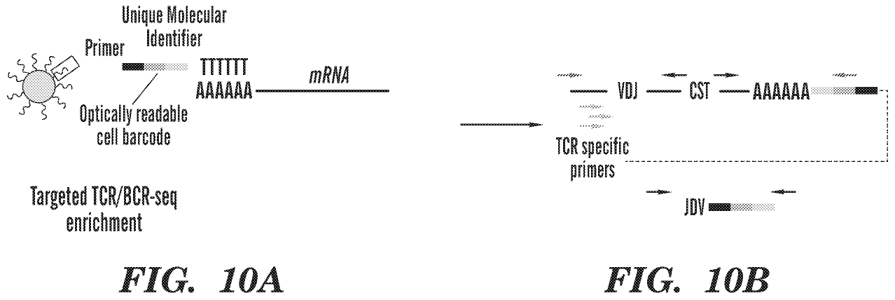

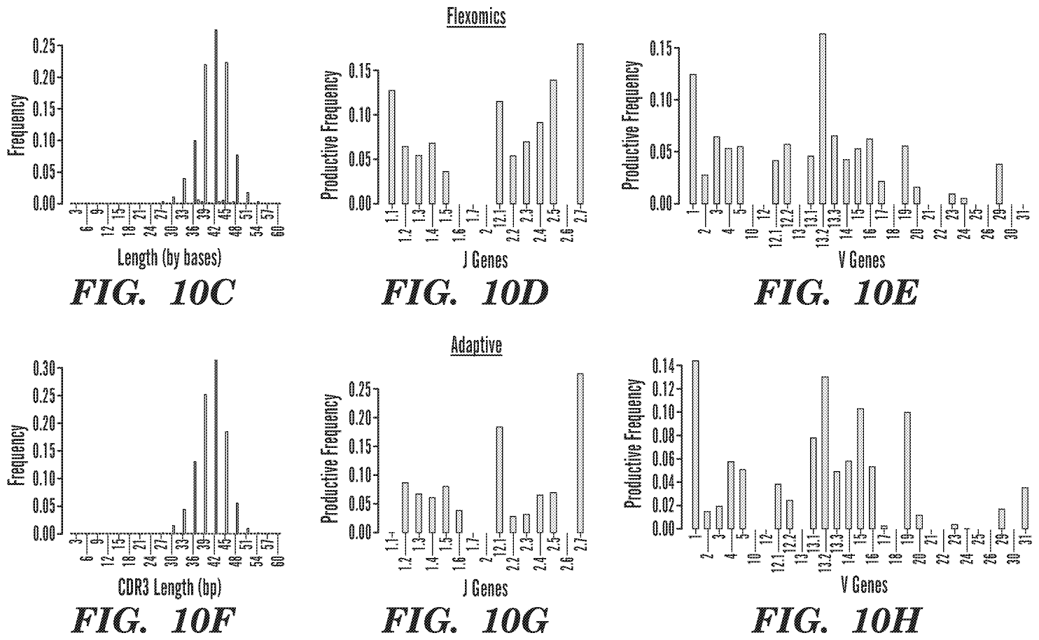

FIGS. 8A-D illustrate one embodiment of the use of capture sequences of the present application. For example, FIG. 8A shows that mRNA sequences can be captured using barcode conjugated oligo-dT capture sequences followed by reverse-transcription and gene specific amplification of barcoded sequences. An exemplary illustration of this process is also shown in FIGS. 9A-B. FIG. 8B shows barcoded capture of T-Cell Receptor or B-Cell Receptor sequences for sequencing. An exemplary illustration of this process is also shown in FIGS. 10A-H. FIG. 8C shows barcoded capture of DNA sequences of interest using UV light to release the primers from the bead.

Nucleotide sequences that form the optically readable oligonucleotide probes and well-identifying barcodes can be built using any method known in the art. An exemplary method includes the split and pool approach scaled-up to generate a sufficient number of barcodes for the tracking of a large number of molecular interactions (see, e.g., Macosko et al., “Highly Parallel Genome-wide Expression Profiling of Individual Cells Using Nanoliter Droplets,” Cell 161:1202-1214 (2015), which is hereby incorporated by reference in its entirety). In some embodiments, nucleotide sequences can be built on beads by combinatorial split and pool approach combined to splint-based DNA ligation of the different words to create combinatorial complexity. See, e.g., Zhang et al., “Haplotype Phasing of Whole Human Genomes using Bead-Based Barcode Partitioning in a Single Tube,” Nature Biotech 35:852-857 (2017), which is hereby incorporated by reference in its entirety. Alternatively, oligonucleotides of specific sequences can be directly synthesized on beads by a split and pool approach as previously described. Ligation based approaches to build the full-length barcodes have the advantage to be easily implemented as they are based on enzymatic reactions in comparison to base by base chemical synthesis that would need to be interrupted after the synthesis of each word to perform the pool and split operation.

In some embodiments, oligonucleotide complexes of the present application form in wells formed on a solid support. The wells are filled with beads to which is conjugated the above described well-identifying barcodes. In some embodiments, each bead comprises a plurality of the same well-identifying barcodes. Through a series of flows, described in more detail infra, the well-identifying barcodes hybridize to complementary nucleotide sequences in optically readable oligonucleotide probes to form optically readable well-identifying barcodes and the oligonucleotide complexes of the present application conjugated to a bead.

Thus, a further aspect of the present application relates to an oligonucleotide-conjugated bead. The oligonucleotide-conjugated bead comprises a bead; a plurality of oligonucleotide complexes conjugated to the bead, where each oligonucleotide complex comprises a 5′ primer sequence; an optically readable well-identifying barcode comprising at least two words, where each word comprises a unique first plurality of nucleotide sequences, where the first plurality of nucleotide sequences are complementary to a second plurality of nucleotide sequences in an optically readable oligonucleotide probe, and where each nucleotide sequence of the second plurality of nucleotide sequences comprises at least 3 distinct fluorescent labels comprising up to 7 unique fluorescent combinations to form each of the fluorescent words; a plurality of unique molecular identifiers; and a plurality of capture sequences.

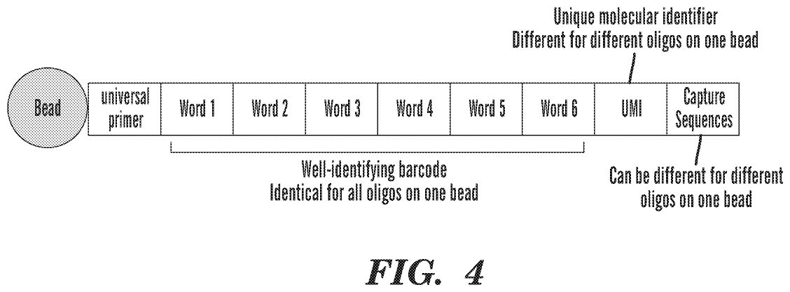

FIG. 4 shows an exemplary illustration of the above-described oligonucleotide-conjugated bead, except having only one 5′ primer sequence, one well-identifying barcode, one UMI, one capture sequence, and no complementary optically readable oligonucleotide probe. Conjugation of oligonucleotides to beads is well known in the art. Exemplary methods of conjugating or otherwise associating oligonucleotides with beads include attachment to the bead with a photo cleavable linker, a linker incorporated to a uracil residue for release by USER (Uracil-Specific Excision Reagent) enzyme digestion, or a restriction site for release by restriction enzyme for release in solution. An oligonucleotide can also be attached to a bead by coating a bead with streptavidin and using a biotinylated oligonucleotide to associate the oligonucleotides and beads.

The beads to which oligonucleotides are conjugated according to the present application can be any shape, including spherical, rectangular, pyramidal, cone-shaped, or any other shape. Exemplary sizes of the beads can range from 0.1 to 100 μm. Bead size may be optimized for the particular solid support platform to be used. For example, to increase bead capture efficiency and reduce bead doublet (i.e., the inclusion of two beads in a single well), bead diameter can be set at ˜½ the size of the well diameter of the solid support. The material of the beads can be made of any suitable material, such as, without limitation, plastic, metal, silica, polysaccharide, or any combination thereof. Exemplary materials include polystyrene, polymethacrylate, polylactic acid, iron oxide, starch, chitosan, silica, and others.

The oligonucleotide complex comprises sequences described above as well as well-identifying barcodes made of a succession of words, each of them being decoded by using successive hybridization of optically readable oligonucleotide probes comprising fluorescent words of known sequences complementary to each barcode word as illustrated in FIG. 3B.

In some embodiments, it is desirable to have a bead conjugated multiple times with the same oligonucleotide to afford hybridization no matter the orientation of the bead on the array. Thus, in one embodiment, the bead is conjugated with a plurality of the same oligonucleotide. In other embodiments, the bead is conjugated with the same well-identifying barcode, but other sequences included in the oligonucleotide with the well-identifying barcode may differ. For example, it may be desirable for each oligonucleotide among a plurality of oligonucleotides conjugated to a single bead to have a different unique molecular identifier. In another example, in some embodiments, the plurality of capture sequences conjugated to a single bead comprise more than one sequence per bead. In some embodiments, the plurality of capture sequences comprise one or more of an oligo-dT sequence, gene specific primer sequences, PCR primers, and combinations thereof.

Another aspect of the present application is directed to a solid support detection system comprising a solid support comprising a plurality of wells and a plurality of oligonucleotide-conjugated beads as described herein, where each of the plurality of oligonucleotide-conjugated beads comprises a unique well-identifying barcode, and where a plurality of the wells comprises one of the oligonucleotide-conjugated beads.

In one embodiment, the well-identifying barcode becomes an optically readable well-identifying barcode when hybridized to an optically readable oligonucleotide probe as described herein.

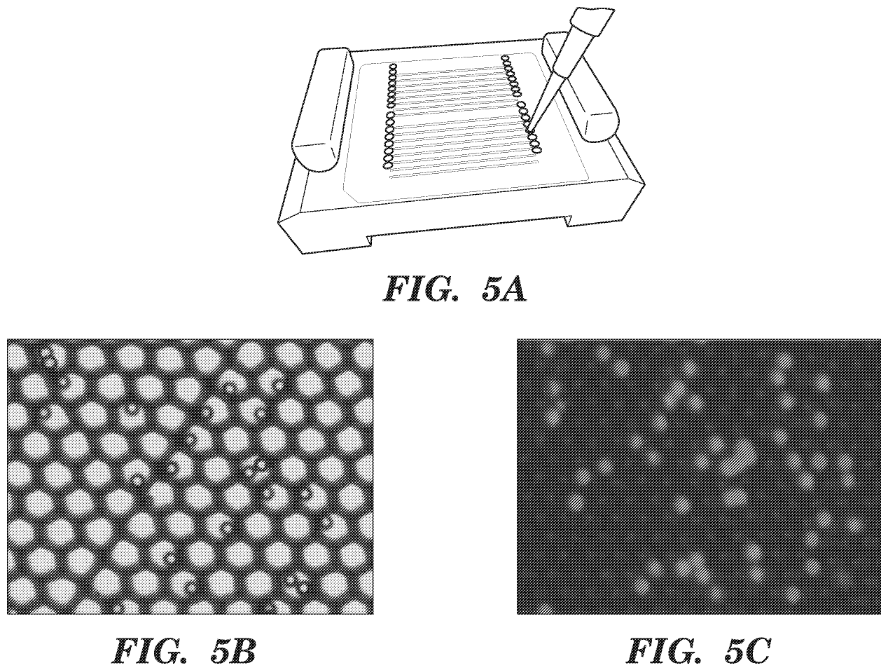

In this aspect of the present application, a solid support comprises a plurality of wells. An exemplary solid support is shown in FIGS. 5A-C as a picowell plate. The solid support of the present application can be used at low throughput with the number of wells ranging from 50 to 2,000 or high throughput with over 10 million wells. In some embodiments, the number of wells is between 50-2000 wells. In some embodiments, the number of wells is between 2,000-10,000 wells. In some embodiments the number of wells is between 10,000-100,000 wells. In some embodiments, the number of wells is greater than 100,000 wells. In some embodiments the number of wells is 100,000-1,000,000 wells. In some embodiments the number of wells is 1 million to 3 million wells. In some embodiments the number of wells is 3 million to 10 million wells. In some embodiments, the number of wells is greater than 10 million wells. One of the unique advantages of the solid support detection system of the present application is the ability to associate the molecular contents of a single well among thousands or even millions of wells on a single solid support from all other wells on that support. This is made possible by the unique structure of the optically readable oligonucleotide probes, each containing multiple possible fluorescent labels forming unique fluorescent combinations to form fluorescent words.

The shape of the wells of the solid support can be round, square, hexagonal, or any other suitable shape. In one embodiment, the well is round on the surface of the solid support and tapers through its depth to come to a point to form a well having a cone-shape. The size of the wells of the solid support can be in the range of 2-100 microns in diameter and 2-100 microns in depth in both dimensions.

The wells of the solid support are made of materials that are characterized by the compatibility with cell viability, functional assay performance, and molecular biology efficiency. The solid support can be made of polydimethylsiloxane (PDMS), glass, fiber optics, polystyrene, polypropylene or any other suitable material. In some embodiments, the solid support can be coated with hylaluronan, polylysine, collagen, or any combination thereof. Such coatings may be used, for example, to promote cell adhesion. In some embodiments, the solid support can be coated with proteins such as streptavidin or antibodies for cell-based functional assays. An example of a solid support is described for example, in Margulies et al., “Genome Sequencing in Microfabricated High-Density Picolitre Reactors,” Nature 15:376-380 (2005), which is hereby incorporated by reference in its entirety. In one embodiment, the well size of the solid support is sufficient for an oligonucleotide-conjugated bead and capture of pairs of cells or cellular components while efficient molecular biology of nucleic acids is determined in picoliter scale reactions.

The loading of barcoded beads, cells, reagents, probes and any other component of this and other aspects of the present application may be loaded onto a solid support (or positioned in picowells of a solid support) by flowing the beads, cells, reagents, probes and any other component onto or across the solid support. The loading device consists of a base, a lid, a gasket and two latches and is assembled as followed. The solid support picowell plate is placed on top of the base, the gasket is added on top of the picowell plate while the lid completes the loading device assembly. Two latches located on each side of the loading device are closed to maintain the base-plate-gasket-lid assembly in place during bead, cell or reagent loading. Alternatively, a microscope slide format of the picowell plate may be loaded using adapted cytology smear techniques and CytoSep™ Cell Funnels for the StatSpin® Cytofuge® (Simport Scientific).

For example, in one embodiment, after loading the beads, the solid support is gently centrifuged to enable the beads to settle into specific wells, then excess supernatant is pipetted out. FIG. 5A is a photograph showing an exemplary solid support high-density solid support. In this example, solid support picowell plates were sourced with well numbers of 1,500,000, well size of 50 μm, and optical properties such as fiber-optic construction preventing fluorescent signal bleed-through between adjacent wells thus allowing maximum well occupancy, efficient fluorescent signal transmission, limited reflection, small 2 mm thickness and etched wells on one side of the fiber faceplate, appropriate for the embodiments of the present application. Exemplary beads were successfully captured in wells as shown in FIG. 5B. In this example, 16 μm polystyrene beads were used. Simultaneous cell and bead capture and co-capturing of bead and cells in picowells is shown by imaging in FIG. 5C with co-capture of fluorescent polystyrene beads (blue) and stained MCF7 cells (red). Similarly to beads, cells are captured on the picowell plate (or in wells of the solid support) by successively pipetting and flowing cells in suspension on top of the surface followed by gentle centrifugation. For example, cells such as T-cells, whether they have been pre-enriched or not for specific features by FACS sorting (for example CD8+ cells), are captured in the wells by flowing a suspension of cells on top of the solid support, centrifuging the plate for the cells to settle in the wells, and removing excess supernatant by pipetting.

In some embodiments, a plurality of the wells further comprise a cell. In some embodiments, a plurality of wells further comprise a component of a cell. In some embodiments, a plurality of wells further comprise a living biological cell. In some embodiments, a plurality of the wells further comprise a non-living biological cell. In some embodiments, the cell is one or more of a T-cell or a B-cell. In some embodiments, the cell is a T-cell. In some embodiments, the cell is a B-cell.

In some embodiments of the solid support detection system, a plurality of wells further comprises, in addition to an oligonucleotide-conjugated bead, an interacting molecular component. As used herein, “interacting” encompasses interactions between cells, interactions between cells and molecules, and interactions between molecules. A “molecular component” comprises whole cells as well as molecules. Molecular components can be added to the wells by successively pipetting and flowing molecular components, which may or may not be attached to beads, on top of the surface of the solid support followed by gentle centrifugation. For example, cells such as Antigen Presenting Cells (“APC”) or antigen coated beads can be loaded by flowing a suspension of beads or cells on top of the device and centrifuging the plate for the cells or a bead to settle in the wells. Excess supernatant is pipetted out. In some embodiments of the solid support detection system, a plurality of the wells comprise an interacting molecular component, and the interacting molecular component is an Antigen Presenting Cell, or an antigen-coated bead.

In some embodiments, the solid support detection system comprises a detectable product from a functional assay for a molecule of interest. For example, an interaction between T-cells and APC cells leads to production of interferon γ (“IFN-γ”), interleukin-2 (“IL-2”), granzyme B, or perforin (as non-limiting examples). This interaction can be detected using fluorescent phenotypic assays. A fluorescently tagged antibody detecting a molecule of interest can be imaged and localized to specific wells. The association of the specific molecular component (i.e., a T-cell) with the output of the fluorescent phenotypic assay is connected through the optically readable well-identifying barcode.

In some embodiments, the solid support system further comprises a cell-lysing agent. For example, a mild lysis reagent may be flowed across the solid support, RNA is captured, barcoded, reverse transcribed, and barcoded cDNA is exported. A harsher lysis buffer can then be brought in for lysis of the nucleus, and DNA can be barcoded, targeted, and amplified using primers carried by either the oligonucleotide-conjugated bead or another bead.

In some embodiments, the solid support detection system further comprises a gasket, where the gasket divides the plurality of wells into separated areas. A gasket placed on top of the surface allows loading distinct samples on distinct areas of the solid support. In some embodiments, the gasket divides the plurality of wells into 2-20 separated areas. In some embodiments, the gasket divides the plurality of wells into 20-1,000 separated areas. In some embodiments, the gasket divides the plurality of wells into greater than 1,000 separated areas. The gasket may be made of any suitable material such as rubber, silicon, polypropylene, flexible polymer, as non-limiting examples. FIG. 7 is an illustration of a gasket separating a solid support into 6 sections. As illustrated, a different solution containing, e.g., 6 different drugs may be screened simultaneously on a single solid support, because the gasket sections of areas of the solid support to isolate groups of wells on the solid support.

A further aspect of the present application is a method for preparing a solid support detection system of the present application, which may be used analyzing molecular interactions or other purposes. This method involves providing a solid support comprising a plurality of wells, and depositing in a plurality of wells a unique, oligonucleotide-conjugated bead. The oligonucleotide-conjugated bead comprises oligonucleotides conjugated to the bead, where the oligonucleotides comprise a 5′ primer sequence, a well-identifying barcode sequence comprising at least two words, where each word comprises a unique first plurality of nucleotide sequences complementary to a probe. The oligonucleotide also comprises a unique molecular identifier and a 3′ capture sequence. The method further involves applying a series of flows, where each flow comprises a set of optically readable oligonucleotide probes as described herein. Each probe comprises a second plurality of nucleotide sequences complementary to the first plurality of nucleotide sequences, where each sequence of the second plurality of nucleotide sequences comprises at least 3 distinct fluorescent labels comprising up to 7 unique fluorescent combinations to form a fluorescent word.

This aspect of the present application can be carried out with any of the embodiments described in this disclosure. In accordance with this and other aspects of the present application, the method involves a series of flows, where each flow comprises a set of optically readable oligonucleotide probes as described herein. Each member of the set of optically readable oligonucleotide probes consists is diluted from a 100 μM stock in duplex buffer (Integrated DNA Technologies, Newark, N.J.). The probe detection solution is introduced on the solid support using the same loading device used for bead loading. The probe detection solution may be exchanged by diffusion for PBS (Thermo Fisher Scientific, Waltham, Mass.) for the imaging and detection of the beads if the solution itself display a high-level of fluorescence.

In one embodiment, the optically readable oligonucleotide probes corresponding to each word of a well-identifying barcode are flowed across the solid support system comprising the oligonucleotide-conjugated beads. Each flow comprises an optically readable oligonucleotide probe comprising a plurality of nucleotide sequences, which anneal to the complementary sequences in the well-identifying barcodes within the oligo-nucleotide conjugated beads located in wells of the solid support. Each well-identifying barcode word is decoded by annealing of the complementary sequence of the optically readable oligonucleotide probe. The complementary optically readable oligonucleotide probes are made of DNA or RNA nucleotides with a size ranging from 6, 7, 8, 9, 10, 11, 12, 13, 14, 15, 16, 17, 18, 19, to 20 by or more and are flowed on a surface of device solid support to diffuse to wells in the solid support. The entire plate may be scanned and pictures taken in 3, 4, 5, 6, or 7 different channels being used for fluorescent detection depending upon the number of fluorescent labels being used.

A fluorescence imaging readout for each individual oligonucleotide-conjugated bead in a well will include a binary output (I/O) for a fluorescent signal in all channels for each flow depending upon the number of fluorescent labels used to make the optically readable oligonucleotide probe. For example, if 3 fluorescent labels were used, the binary output would have three elements (e.g., Flow 1: “100” equivalent to positive in fluorescent channel 1 and negative in channels 2 and 3, in Flow 2: “011”, equivalent to negative in fluorescent channel 1 and positive in channels 2 and 3, etc.). If 4 fluorescent labels were used, the binary output would have a four element readout for each individual bead in a well for the fluorescent signal in all 4 channels for each flow (e.g., Flow 1: “1001” equivalent to positive in fluorescent channel 1 and 4 and negative in channel 2 and 3, Flow 2: “0111”, etc.). Analysis of the fluorescent signals in each position or well on the solid support produces a map of well positions and a list of registered signals that allows the reconstitution of the nucleotide sequence that is present on each individual bead. The positional information of each well-identifying barcode in the solid support detection system, when decoded by flowing and imaging fluorescent words, becomes a map, where the position of each bead on the solid support is known. Thus, in one embodiment, the present application is directed to a solid support pre-loaded with well-identifying barcoded oligonucleotide-conjugated beads to which a barcode map identifies which barcoded bead is present in which well. In one embodiment, the solid support detection system further comprises a map identifying the location on the solid support, by well, of each oligonucleotide-conjugated bead.

Accordingly, in some embodiments, the method further comprises imaging the solid support to identify wells containing beads with a fluorescent word. In one embodiment, the position and sequence of each bead is decoded by imaging the fluorescent signals in 3 different channels for each individual well corresponding to the three fluorescent labels used to label the optically readable oligonucleotide probe. In some embodiments, the fluorescent signal is imaged in 4 different channels for each individual well corresponding to the 4 fluorescent labels used to label the optically readable oligonucleotide probe. In some embodiments, the fluorescent signal is imaged in 5 different channels for each individual well corresponding to the 5 fluorescent labels used to label the optically readable oligonucleotide probe. In some embodiments, the fluorescent signal is imaged in 6 different channels for each individual well corresponding to the 6 fluorescent labels used to label the optically readable oligonucleotide probe. In some embodiments, the fluorescent signal is imaged in 7 different channels for each individual well corresponding to the 7 fluorescent labels used to label the optically readable oligonucleotide probe. In some embodiments, the imaging is carried out after each flow in the series. In some embodiments, the method comprises 6 series of flows, where each flow comprises a set of the optically readable oligonucleotide probes. In some embodiments, the method comprises 7, 8, 9, 10, 11, 12, 13, 14, 15, 16, 17, 18, 19, 20, or more than 20 series of flows, where each flow comprises a set of the optically readable oligonucleotide probes.

Fluorescent signal generated by oligonucleotide complexes and functional assays performed in each well may be collected by fiber optic like-properties of the solid support plate and measured via a standard inverted microscope. Other solid supports are contemplated that do not use fiber optics. Between flows, fluorescent signals can be eliminated by photobleaching or by release of the probes from the beads via photocleavage of a photocleavable linker, restriction site digestion, uracil digestion by USER (Uracil Specific Excision Reagent) enzyme, or denaturation in the presence of NaOH. In some embodiments, the method further comprises eliminating fluorescence between each flow.

An analysis package linking imaging based functional data and sequence information may also be used in this and other methods of the present application. In one embodiment, the analysis package may include the basic components for manual screening of a small number of molecular interactions and can be scaled up using automation. The system may include a processor, a memory, a database, and an application stored in the memory that when executed on the processor, can perform the following steps. The software application package can spatially identify and link optically labeled beads (e.g., oligonucleotide complexes described herein) and cell based assay readouts on a high-density solid support picowell (>10⁶ wells) array. The software application can also identify wells containing no cell, one cell, two cells, or more than 2 cells. The package can include a fluorescent image acquisition module, image processing programs capabilities module (image stitching for full solid support screening, fluorescent detection, and mapping), data processing, and a user interface.

Beads and molecular components are captured on the solid support by successively pipetting and flowing cells on suspension on top of the surface of the solid support followed by gentle centrifugation as discussed supra. The successive loading of beads, cells, molecular components and assays leads to the co-capture of barcoded beads, assay cell/bead and assayed cell. In some embodiments of the method, a plurality of the wells further comprise a cell. In some embodiments of the method, the cell is one or more of a T-cell or a B-cell. In some embodiments of the method, a plurality of the wells further comprise an interacting molecular component. In some embodiments of the method, a plurality of the wells comprise an interacting molecular component and the interacting molecular component is an Antigen Presenting Cell. In some embodiments of the method, a plurality of the wells comprise an interacting molecular component and the interacting molecular component is an antigen.

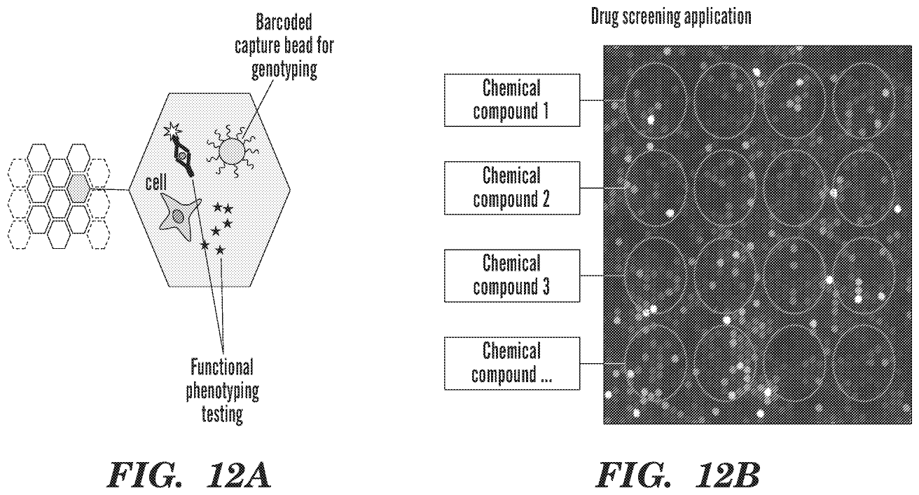

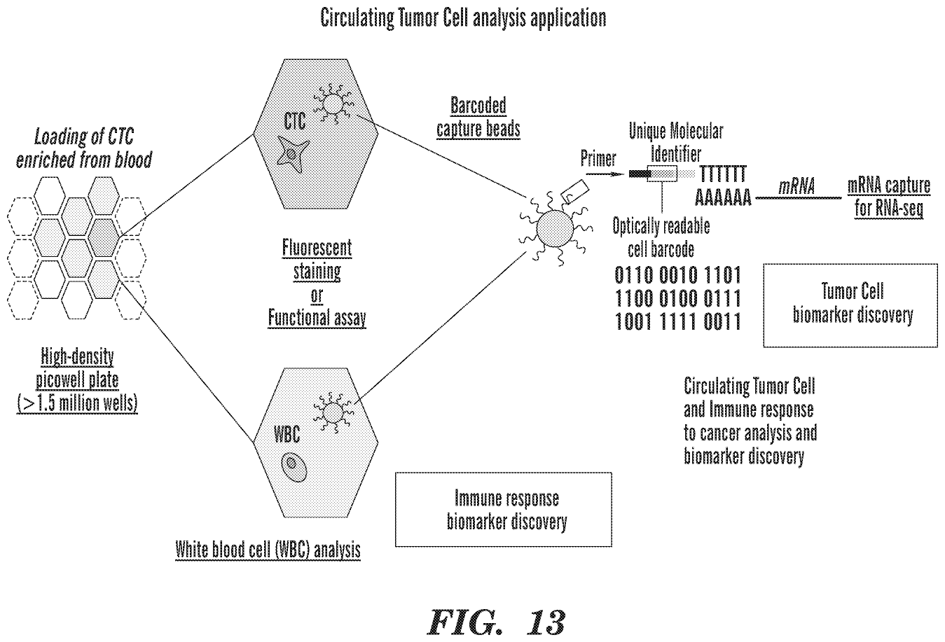

In some embodiments, the method further comprises applying a gasket, where the gasket divides the plurality of wells into separated areas. In some embodiments the gasket divides the plurality of wells into 2-20 separated areas. In some embodiments the gasket divides the plurality of wells into 20-1000 separated areas. In some embodiments the gasket divides the plurality of wells into more than 1,000 separated areas. In some embodiments the method of this and other aspects of the invention further comprises applying a chemical compound to the separated areas. FIG. 7 and FIG. 12 provide an exemplary illustration of this embodiment of the present application. In FIG. 7, different areas of the solid support are separated by a gasket. Each separated area can be exposed to different chemical compounds or drugs to allow single cell response measurement for a high-number of cells and a high number of chemical compounds in parallel (see also FIG. 12). This single cell resolution is distinct from the usual/conventional methods in which chemical compounds or drugs are applied to a bulk of cells and an average response is measured. This ability to query at the single cell level becomes specifically important when looking at primary cells compared to cell lines. In the context of drug screening, a person of ordinary skill in the art would appreciate that the method of the present application can be utilized to submit cells to a first set of compounds, image and characterize phenotypically the cell response, then add another compound and do this for several cycles that may include cell culture periods. At the end, the cells are lysed for capture of RNA and/or DNA.

In some embodiments, this and other methods of the present application further comprise culturing the cells on the solid support. Culturing the cells as growing colonies can help to improve the quality of the data captured. Culturing can include perfusing the cells in the solid support with media and CO₂ and incubating at 37° C. to promote cell growth and viability, and maintaining a layer of media in the loading channel of the loading device that is common to all wells to maintain cell to cell communication, as non-limiting examples.

In some embodiments, this and other methods of the present application further comprise detecting a product from a functional assay for a molecule of interest. Functional assays can be used to detect, for example and without limitation, proliferation, cytokine secretion, or cytolytic assays. For example, cell response to a given antigen can be measured by fluorescent microscopy and fluorescent signal of a given well corresponding to a functional response for the cell captured in that specific well. Similar to barcoded bead detection, the solid support can be screened in different fluorescent channels, each of them corresponding to one readout of a multiplex assay (e.g., IL-2 secretion measured in GFP channel, IFN-γ measured in TexasRed channel) and the final results producing a map identifying which well contains activated T-cell or other cells of interest for other applications. The assays can be commercially available gold-standard assays such as immunofluorescence (e.g. fluorospot), for example, miniaturized for compatibility with the solid support system of the present application. The assay can also be a colorimetric assay. Similar to bead decoding, fluorescent based functional assays can be performed by flowing different types of reagents and cell response is recorded for each individual cell or pair of cells by well position. Finally, bead identification through the well-identifying barcode and cell-based assay readouts can be linked based on spatial mapping. In some embodiments, a functional assay might include cell culturing.

In some embodiments, this and other methods of the present application further comprise lysing the cells. Lysis buffer such as detergent-based or proteinase K based can be flowed on the surface of the plate so that it diffuses into the wells to lyse the cells. For a mild lysis buffer, only the cytoplasm breaks-open and released RNA can be captured on the barcoded beads via either oligo-dT for the capture of mRNA or gene-specific primers for targeted capture of specific genes. A harsher lysis buffer can also be used instead of or in a subsequent step to break open the nucleus and free genomic DNA that can also be released.

In some embodiments, this and other methods of the present application further comprise detecting nucleotide sequences from the molecular components containing nucleotide sequences. Once barcoded beads have been mapped and cells of interest have been identified by position, RNA and/or DNA can be accessed, captured, and barcoded via the oligonucleotides bound to the barcoded beads. FIG. 8 illustrates the different types of capture primers that can be carried and potentially delivered by the beads: oligo-dT for mRNA capture, gene specific primer for reverse transcription for targeted RNA-sequencing (e.g., constant region for T-cell Receptor (TCR) and B Cell Receptor (BCR) sequencing), PCR primer for DNA enrichment, and mutation detection. The system further includes a sequencing-based assay to determine TCR-sequences, epitopes, and gene expression profiles, which incorporates a sequencing library method that encompasses the decoding of the phenotypic barcode, RNA-sequencing for complementary functional information, and TCR and epitope sequences.

For DNA information capture, barcoded primers specific of regions of interest can be released from the beads via photocleavage of photocleavable linker, restriction site digestion, or uracil digestion by USER (Uracil-Specific Excision Reagent) enzyme and PCR based or isothermal amplification that is carried out on the solid support (e.g., in the wells). Genomic DNA can be fragmented and tagged in the well by using in solution or on-bead Tn5 or alternative transposase. The barcoded beads can then carry universal barcoded primers for the barcoding of genomic DNA fragments on the solid support (e.g., in the wells).

Resulting barcoded whole transcriptome cDNA, targeted cDNA, whole genome fragments or targeted DNA amplicon molecules diffuse out in a collection buffer such as Tris-EDTA that is flowed on top of the solid support and pipetted out for retrieval or RNA and/or DNA information in a pooled format. The extraction part involving diffusion of material of interest can be repeated several times to increase the recovery yield. Retrieval can also be facilitated via photocleavage of a photocleavable linker, restriction site digestion, uracil digestion by USER (Uracil Specific Excision Reagent) enzyme, or denaturation in the presence of NaOH. An alternative to extract material captured on beads consists in retrieving the beads themselves, which could be achieved by using magnetic beads and with a magnet applied on the top of the device to suspend the beads above the wells for retrieval.

Extracted barcoded material is subjected to final Next Generation library sequencing and a second set of barcode or index can be added by PCR or ligation to increase the combinatorial number of samples that can be multiplexed for sequencing and subsequently identified. The sequencing may involve identifying both the RNA or DNA information, but also the well-identifying barcode identifying the position in the plate from which the material originated. In all cases, the well-identifying barcode on the oligonucleotide with the capture sequence allows the linking of information from the mapped position of cells and their phenotypic assay results with genotypic information on the cells in that well.

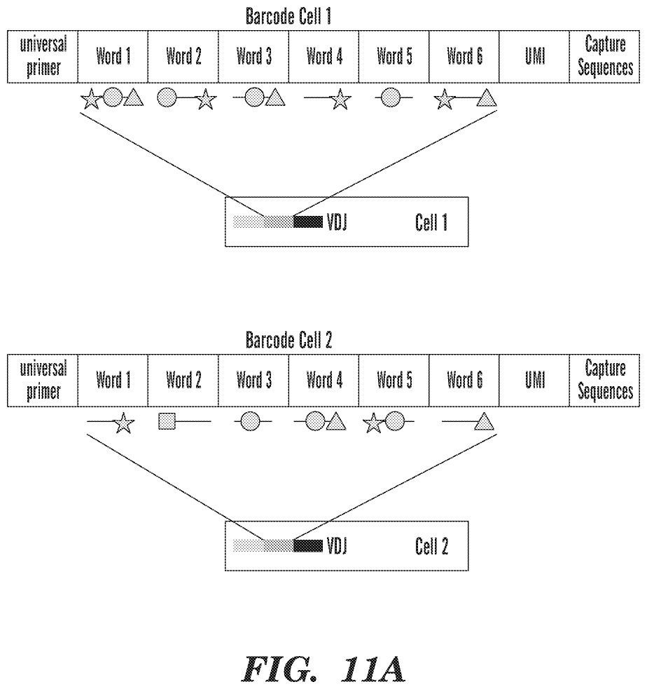

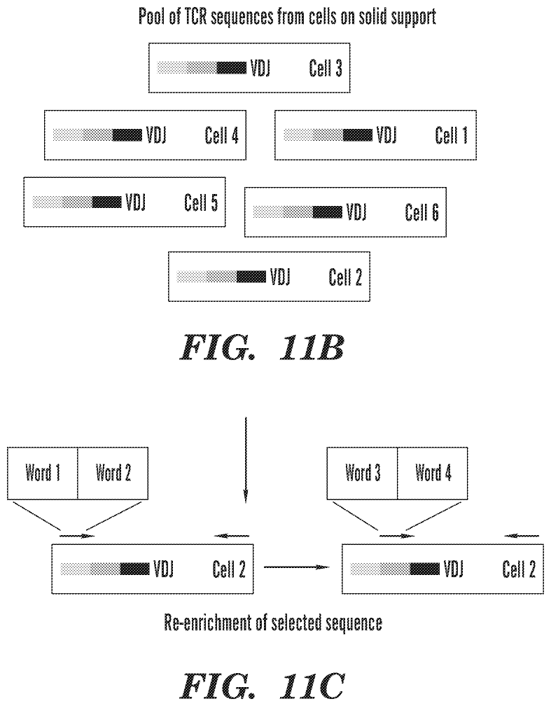

In some embodiments, this and other methods of the present application further comprise re-enriching sequences from target wells using primers, where one primer comprises the well-identifying barcode. Because the barcodes are designed to be of known sequence and are located on the 3′ end side of captured transcripts, it is possible to use barcoded specific primers in conjunction with universal primers located on 5′ (e.g. complementary to the template switching oligo) to re-enrich a specific amplicon (e.g. a T-cell receptor from a specific cell from the barcoded pooled cDNA) for downstream cloning and functional validation. FIGS. 11A-C illustrate the method of the present application for the enrichment of transcript/sequence from specific cells from the pooled cDNA and/or DNA but re-using the combination of a primer comprising the well-identifying barcode in a nested PCR to target the molecules that carry these specific sequences. As an example, TCR or BCR sequences can be enriched using multiplexed forward primers covering the diversity of sequence. Full length barcoded cDNA can be circularized by T4 ligation and library insert size can be reduced by PCR to remove the constant region while keeping the barcoded VDJ region allowing its sequencing using the Illumina technology (2×300 bp reads). Alternatively, the full-length barcoded cDNA for TCR and BCR can be sequenced with any long-read technology, e.g., PacBio SMRT or Oxford Nanopore technologies. This unique embodiment of the present application eliminates the need to resynthesize and clone sequences of interest such as T-cell receptors or B-cell receptors as non-limiting examples. Instead, these sequences are rapidly available for immediate use in downstream applications such as cloning of TCRs for the transgenic expression of antigen-specific TCR and functional validation of promising candidates without the need to fully re-synthesize the full TCR sequence.

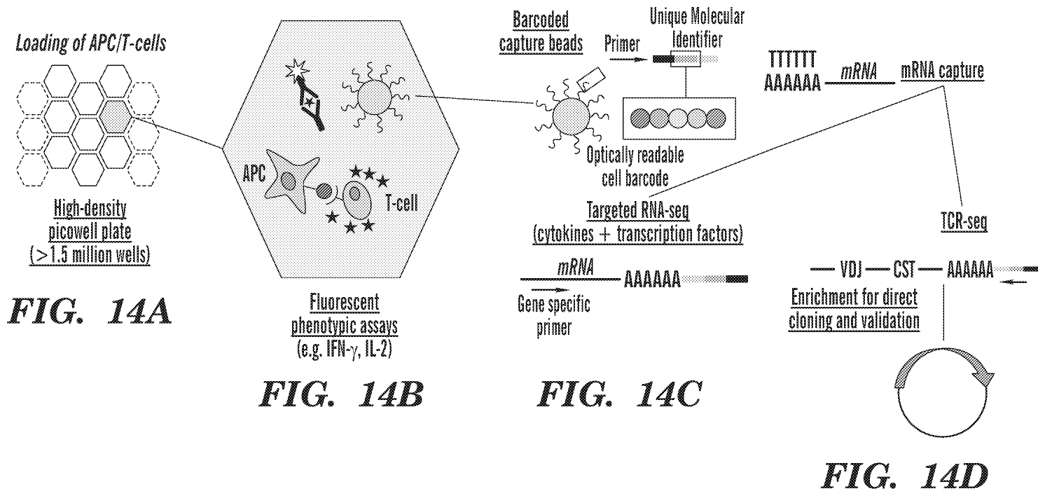

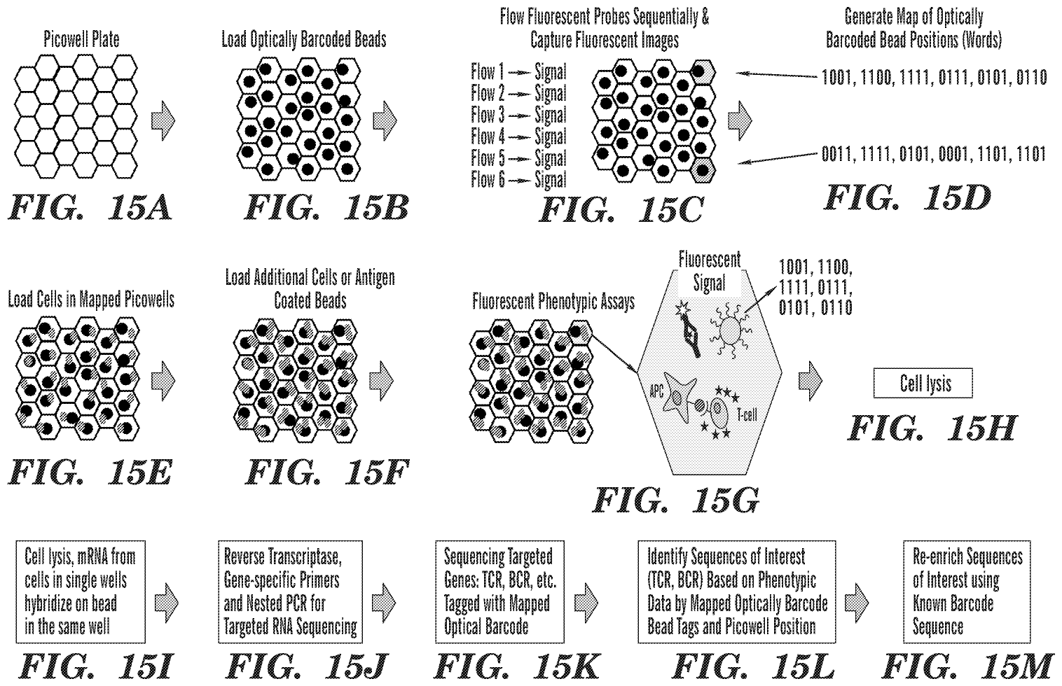

FIG. 14 illustrates an antigen-specific TCR characterization workflow of the present application. The system comprises a high-density solid support having a plurality of wells (in some instances, several millions wells), which allows interrogating >100,000 cells with a simple loading method (FIG. 14A). Fluorescent signal generated by functional assays performed in each well can be imaged and measured via either an upright or inverted microscope. If an interaction between the T-cells and APC cells or antigens leads to production of interferon γ (“IFN-γ”), interleukin-2 (“IL-2”), granzyme B, or perforin (as non-limiting examples), this interaction can be detected using fluorescent phenotypic assays. T-cells and antigen-presenting cells (“APC”) loaded into a well of the solid support are illustrated as well as a fluorescently tagged antibody detecting a molecule shown in the upper left corner of the well (FIG. 14B). The association of the specific wells associated with the fluorescent phenotypic assay is through the optically readable barcode (“Barcoded capture beads”). The system also incorporates a well-identifying barcoding system linking phenotypic and genotypic information, which is a system of nucleic-acid based barcodes that can be detected both by fluorescence and by sequencing (FIG. 14C). Each well-identifying barcode is designed as a succession of “words” (illustrated by a concatemer of differently shaded circles) and is decoded using successive hybridization of fluorescent probes of known sequences complementary to each “word” linking each antigen-T-cell response to nucleic acids captured on the same bead. The method includes a split and pool approach scaled-up to generate a sufficient number of barcodes for the tracking of a large number of antigen-TCR interactions. The system further includes a sequencing-based assay to determine TCR-sequences, epitopes and gene expression profiles, which incorporates a sequencing library method that encompasses the decoding of the phenotypic barcode, RNA-sequencing for complementary functional information, and TCR and epitope sequences. A specific amplicon (e.g. a T-cell receptor from a specific cell from the barcoded pooled cDNA) can be recovered for downstream cloning and functional validation (FIG. 14D and FIGS. 11A-C).

Another aspect of the present application is a high-throughput method of analyzing molecular interactions. This method involves providing the solid support detection system of the present application and contacting the solid support with molecular components to be analyzed, where the molecular components are deposited in the plurality of wells. The method further comprises detecting phenotypic interactions between the molecular components, capturing RNA or DNA derived from the molecular components, transcribing captured RNA to DNA, enriching the DNA, sequencing the DNA, and matching sequences from the sequenced DNA to barcode sequences to identify the well location and, therefore, molecular interactions of interest among the molecular components.

This aspect of the present application can be carried out with any of the compositions, systems, and methods described in this disclosure.

### EXAMPLES

### Example 1—Barcode Building

A test barcode composed of two words was built on 16 μm polystyrene beads coated with streptavidin. The oligonucleotide containing word 1 (bold and underlined in SEQ ID NO:1 below) was designed with a biotin at the 5′ end, a universal primer for amplification and an additional 4 nucleotides (CCGC) following word 1 on the 3′end extremity (Word 1 Oligonucleotide, SEQ ID NO:1 /5BiosG/ACACTCTTTCCCTACACGACGCTCTTCCGATCTCCTCAACATACCGC). A 5′ phosphorylated oligonucleotide designed as the reverse complement to word 1 (Word 1 Reverse Complement, SEQ ID NO:2/5Phos/TATGTTGAGG) was resuspended in duplex buffer (Integrated DNA Technologies, Newark, N.J.) and annealed to the first oligonucleotide to create a duplex with a 4 nucleotide 3′ overhang suitable for ligation. Similarly, another double-stranded oligonucleotide containing word 2 (bold and underlined, SEQ ID NO:3) was designed and annealed to form a 3′ overhang on the opposite strand to complement the 3′ overhang from the oligonucleotide containing word 1 (Word 2 Oligonucleotide, SEQ ID NO:3 /5Phos/\sqrt{TGTCCGAAAG}NNNNNNNNNNTTTTTTTTTTTTTTTTTTTTVN and Word 2 reverse complement, SEQ ID NO:4 CTTTCGGACAGCGG). In this example, the oligonucleotide containing word 2 (SEQ ID NO:3) also contains a 10-mer Unique Molecular Identifier (UMI) and an anchored oligo-dT sequence for mRNA capture (capture sequence).

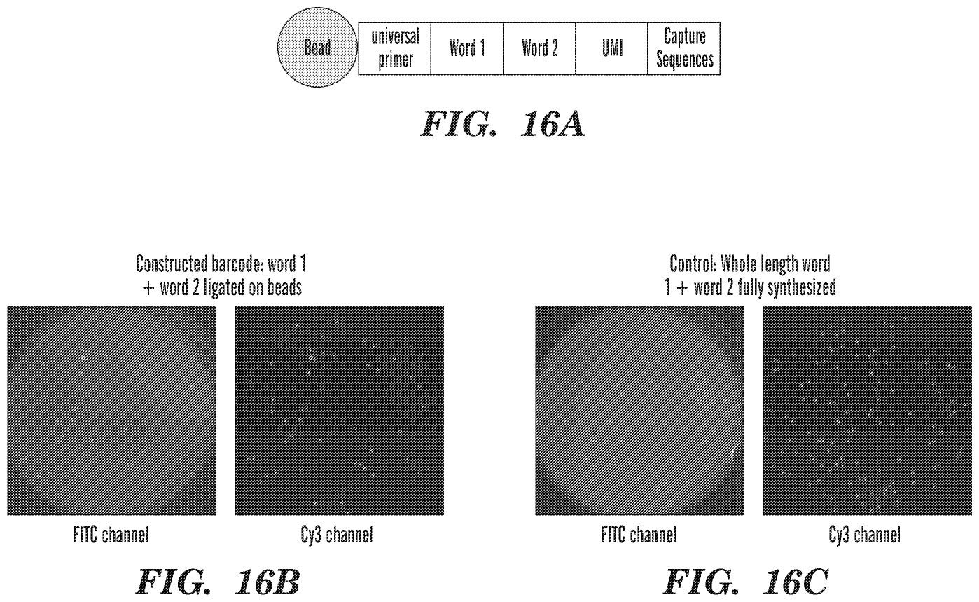

The beads were washed twice in 500 μl of washing/binding buffer (20 mM Tris pH 7.5, 1M NaCl, 1 mM EDTA, 0.0005% Triton® X-100) and the duplex biotinylated oligonucleotide containing word 1 was attached to the beads by adding 1 μl of 100 μM oligonucleotides to 50 μl of washed beads and vigorously mixed on a vortex for 15 minutes at room temperature. The beads were washed twice in 500 μl of washing/binding buffer and resuspended in 9 μl of nuclease-free water. The word 2 duplex oligonucleotide was ligated onto word 1 oligonucleotide immobilized on beads by adding 1 μl of the word 2 oligonucleotide at 100 μM and 10 μl of Instant Sticky-end Ligase Master Mix (New England Biolabs, Ipswich, Mass.) to the beads and incubating for 10 minutes at room temperature. Following the ligation, beads were washed twice with 500 μl of washing/binding buffer. Resulting barcoded beads were incubated for 5 minutes at room temperature in presence of 0.1 M NaOH to denature and remove the complementary strand not physically attached to the beads. The oligonucleotide-conjugated bead is illustrated in FIG. 16A.

Full barcodes comprising word 1 and word 2 were visualized on a fluorescent upright microscope by incubating the beads with a 1/100 dilution of complementary fluorescent word DNA probes labeled with either Cy3 for word 1 (SEQ ID NO:2 TATGTTGAGG/3Cy3Sp/) labeled on the 3′ end with Cy3or Alexa488 for word 2 (Word 2 Fluorescent Probe, SEQ ID NO:5 CTTTCGGACA/3AlexF488N/) labeled on the 3′ end with Alexa488 in duplex buffer (Integrated DNA Technologies, Newark, N.J.) for 2 minutes at room temperature. Imaging of the constructed oligonucleotide-conjugated bead complexed with the fluorescent probes is shown in FIG. 16B.

As an alternative, the same constructed sequence was fully synthesized chemically by IDT (Integrated DNA Technologies, Newark, N.J.). The oligonucleotide contained Word 1 and Word 2, UMI, and an oligo-dT capture sequence (Oligonucleotide Word 1 and 2, SEQ ID NO:6 /5Biosg/ACACTCTTTCCCTACACGACGCTCTTCCGATCTCCTCAACATATGTCC GAAAGNNNNNNNNNNTTTTTTTTTTTTTTTTTTTTVN) and tested side-by-side with probes to words 1 and 2 (SEQ ID NOs:2 and 5) as a positive control. Imaging of the synthesized oligonucleotide-conjugated bead complexed with the fluorescent probes is shown in FIG. 16C.

A high number of combinations of oligonucleotides with words is achieved by a split and pool approach applied to the different words composing all possible barcodes. The split approach can be applied during direct chemical synthesis on beads with pre-determined word sequences synthesized in each pool or during the ligation step, whether it be via sticky-end (Instant Sticky-end Ligase Master Mix, New England Biolabs, Ipswich, Mass.), blunt-end (T4 Ligase, Thermo Fisher Scientific, Waltham, Mass.) or splint-based ligation (SplintR, New England Biolabs, Ipswich, Mass.), of different sets of words in each pool following the splitting step. Barcoded oligonucleotides may be attached and built on the beads by biotin-streptavidin interactions but also other non-covalent and covalent interactions.

### Example 2—Plate Loading Architecture

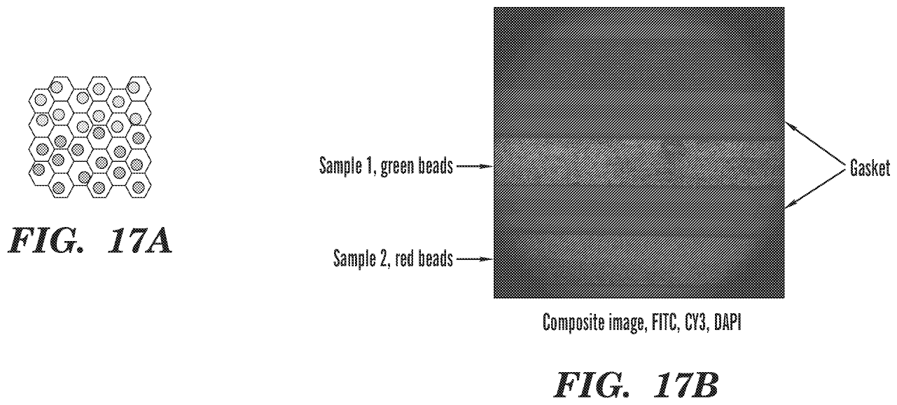

A prototype loading device comprising a holder, a 75×50 mm solid support containing >1.5×10⁶˜30 μm wells, and a gasket dividing the picowell in 16 distinct sample-specific channels is shown in FIG. 5A. Two of these distinct channels were loaded with either Dragon Green Fluorescent Microspheres or Suncoast Yellow Fluorescent Microspheres (Bangs Laboratories Inc., Fishers, Ind.) illustrated in FIG. 17A. The microspheres were diluted 1/4 in Phosphate Buffer Saline (Thermo Fisher Scientific, Waltham, Mass.) and 120 μl of the diluted microspheres were flowed over the solid support by introducing the bead dilution through the built-in holes of the prototype loading device as shown in FIGS. 17A-B. The whole device was then imaged under a fluorescent stereo microscope and the microspheres were detected in the GFP and RFP channels and merged into a composite image (FIG. 17B). An enlarged view of the picowells is shown in FIG. 5B, in which polystyrene beads of 16 μm have been successfully loaded. Co-capture in picowells FluoSpheres Polystyrene Microspheres, 15 μm, blue fluorescent (365/415) (Thermo Fisher Scientific, Waltham, Mass.) and MCF7 cells stained with CellTracker™ Red CMTPX Dye (Thermo Fisher Scientific, Waltham, Mass.) is shown in FIG. 5C.

### Example 3—Barcoded Bead Detection, Decoding, and Probe Generation

Barcoded oligonucleotide-conjugated beads were detected using fluorescent probes that were internally labelled with fluorescent nucleotides and complementary to word 1. RNA probes were generated from a single strand DNA template oligonucleotide that includes the word sequence as well as a T7 promoter (T7 Word 1 oligonucleotide, SEQ ID NO:7 AAATGTCCCGCCTGTCTC; Integrated DNA Technologies, Newark, N.J.). The single strand DNA template was converted into dsDNA via a Klenow DNA Polymerase which was then subject to T7 RNA polymerase based transcription in presence of a fluorescein RNA labeling Mix (Roche, South San Francisco, Calif.) according to the manufacturer's protocol (mirVana miRNA Probe Construction Kit, Thermo Fisher Scientific, Waltham, Mass.) to generate fluorescent RNA probes complementary to the word to be detected. The DNA template was removed by digestion with DNase I according to the manufacturer's protocol and resulting fluorescently labelled RNA probes were diluted at 1/100 in duplex buffer (Integrated DNA Technologies, Newark, N.J.).

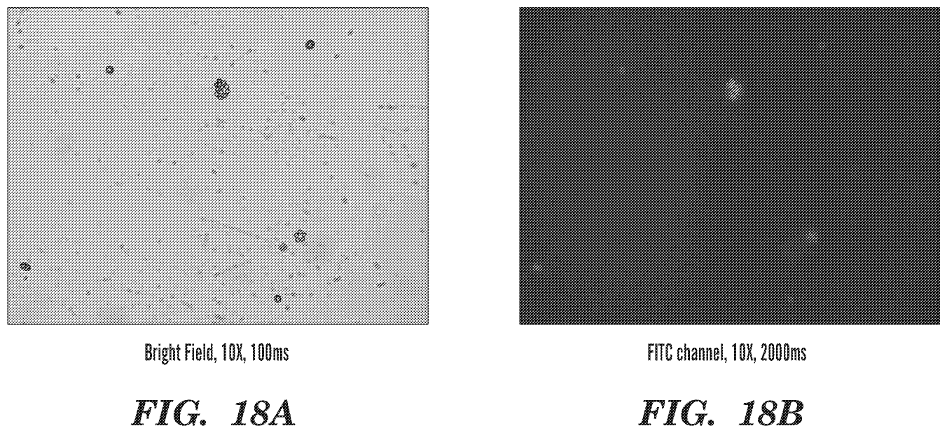

FIGS. 18A-B are photographic images of oligonucleotide-conjugated beads with barcode words visualized by hybridizing the oligonucleotide-conjugated bead with the fluorescently labelled, complementary probes generated by a T7 polymerase starting with a single stranded template (bright field (FIG. 18A) and FITC fluorescent channel (FIG. 18B)).

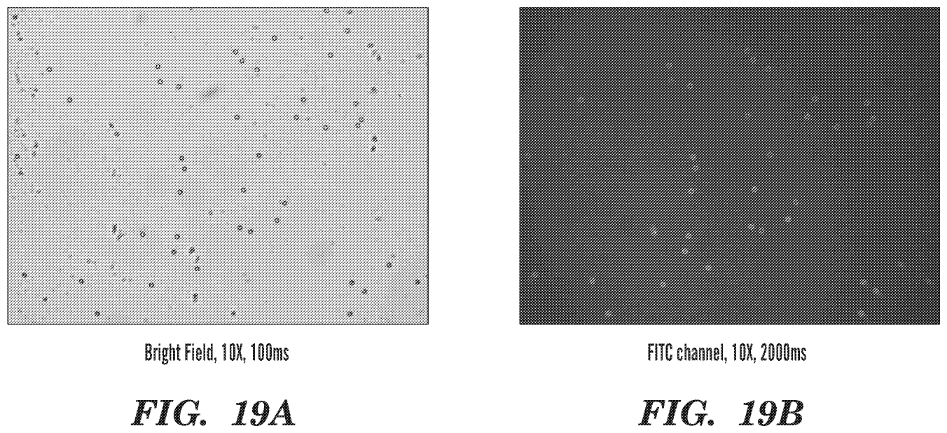

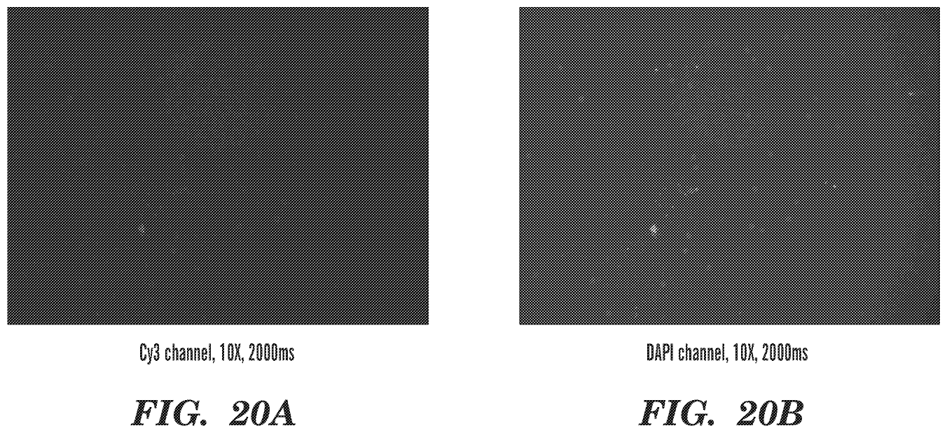

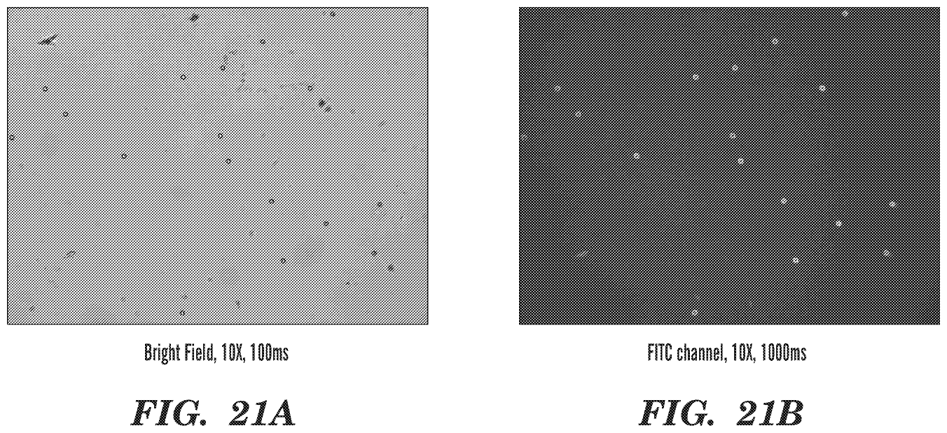

A similar set of fluorescent internally labelled RNA probes was generated starting from synthesized and annealed duplex DNA template carrying either a T7 promoter (T7 Word 1 duplex oligonucleotide, SEQ ID NO:8 TAATACGACTCACTATAGCGCGGGACATTT, INTEGRATED DNA TECHNOLOGIES) or a SP6 promoter (SP6 Word 1 duplex oligonucleotide, SEQ ID NO:9 ATTTAGGTGACACTATAGCGGGACATTT, INTEGRATED DNA TECHNOLOGIES). Transcription was carried using either a T7 or a SP6 RNA polymerase (New England Biolabs, Ipswich, Mass.) respectively and followed by DNase I (New England Biolabs, Ipswich, Mass.) treatment according to the manufacturer protocols. The T7 generated internally fluorescently labelled probes generated from a duplex template and hybridized to a oligonucleotide-conjugated bead with a complementary barcode word are shown in FIGS. 19A-B (bright field (FIG. 19A) and FITC fluorescent channel (FIG. 19B)). FIGS. 20A-B show that there is no detection of the probe from FIGS. 19A-B in the Cy3 channel, and limited bead auto-fluorescence is detectable in the DAPI channel. The SP6 generated internally labelled probes generated from a duplex template and hybridized to a oligonucleotide-conjugated bead with a complementary barcode word are shown in FIGS. 21A-B (bright field and FITC fluorescent channel).

### Example 4—Multicolor Based Barcoded Bead Decoding

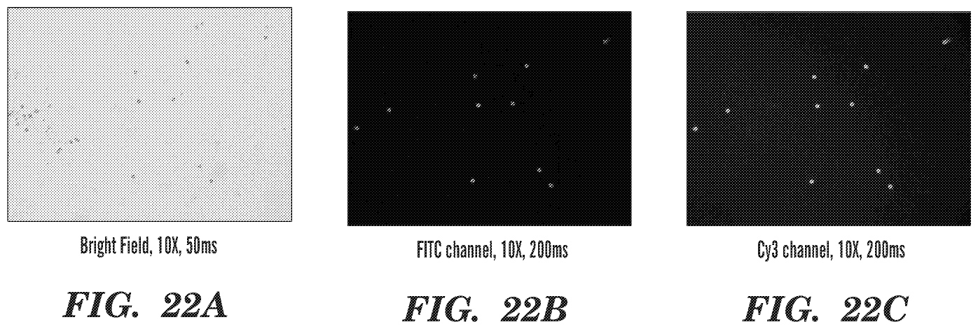

Fluorescent probes may be labelled in different channels, increasing the number of probes that can be pooled and detected simultaneously in the solid support. Dually encoded fluorescent probes (SEQ ID NO:2) labelled in the green channel on 5′ and in the red channel on 3′ (/5Alex488N/TATGTTGAGG/3Cy3Sp/, Integrated DNA Technologies, Newark, N.J.) were used to detect beads coated with oligonucleotides with the complementary word in the FITC and Cy3 channels. FIGS. 22A-C show the oligonucleotide-conjugated bead complex with the fluorescent probe in brightfield (FIG. 22A), FITC channel (FIG. 22B) and Cy3 channel (FIG. 22C).

### Example 5—Barcoded Beads Loading, Deciphering, and Mapping on a Solid Support

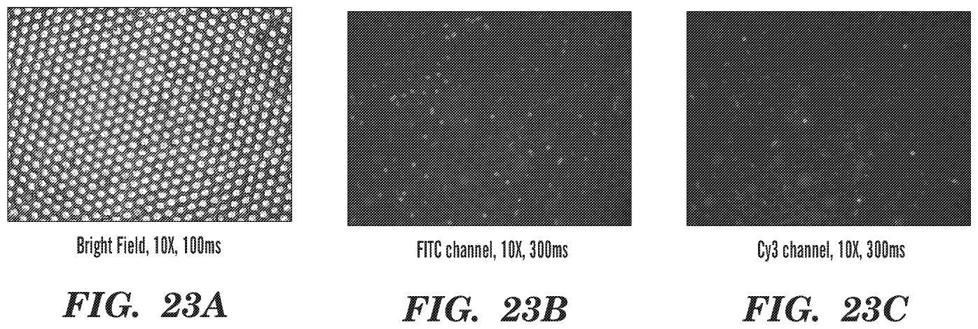

A mixture of two types of 16 μm oligonucleotide-conjugated beads, one coated with an oligonucleotide with the word 1 barcode and the other oligonucleotide carrying the word 2 barcode were loaded on a solid support (FIG. 23A). A mixture of fluorescently labelled probe 1 complementary to word 1 (SEQ ID NO:10 CGGGACATTT/3AlexF488N/, Integrated DNA Technologies, Newark, N.J.) and probe 2 complementary to word 2 (SEQ ID NO:2 TATGTTGAGG/3Cy3Sp, Integrated DNA Technologies, Newark, N.J.) were diluted 1/100 in duplex buffer (Integrated DNA Technologies, Newark, N.J.) and flowed onto the solid support using the same loading device used for bead loading. Oligonucleotide-conjugated beads and probes were incubated for 2 minutes at room temperature to allow complex formation and the solid support was imaged in the FITC (FIG. 23B) and Cy3 (FIG. 23C) channels. Barcode bead types were mapped on the solid support based on fluorescent signal detected in each channel for each bead.

### Example 6—Cell Loading of the Solid Support

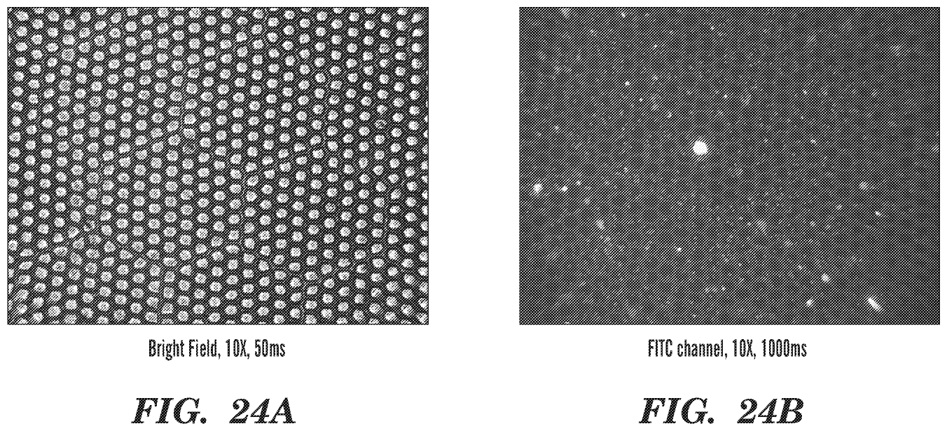

Jurkat cells (T-cells, ATCC) were stained with CellTrace CFSE, cell proliferation kit (Thermo Fisher Scientific, Waltham, Mass.) according to the manufacturer's protocol, harvested, washed with PBS (Thermo Fisher Scientific, Waltham, Mass.) and resuspended at a concentration of ˜3×10⁶ cells/mL. About 300,000 cells were loaded in one of the 16 channels of a solid support by flowing ˜100 μl over the solid support (FIG. 24A). The solid support was centrifuged for 5 minutes at 300 g and imaged under a fluorescent upright microscope in the GFP channel. Cells were detected and located in the wells of the solid support based on their fluorescent signals (FIG. 24B). Cell concentration may be adjusted prior to loading to target specific well occupancy.

### Example 7—Co-Capture of Barcoded Beads and Different Cell Types

A mixture of 16 μm oligonucleotide-conjugated beads with barcodes comprised of either word 1 and word 2 (SEQ ID NO:11 /5BiosG/ACACTCTTTCCCTACACGACGCTCTTCCGATCTAAATGTCCCGGGGTT ATAGANNNNNNNNNNTTTTTTTTTTTTTTTTTTTTVN, Integrated DNA Technologies, Newark, N.J.) or word 3 and word 4 (SEQ ID NO:12 /5BiosG/ACACTCTTTCCCTACACGACGCTCTTCCGATCTCCTCAACATATGTCC GAAAGNNNNNNNNNNTTTTTTTTTTTTTTTTTTTTVN, Integrated DNA Technologies, Newark, N.J.) were incubated with a Cy3 fluorescently labelled complementary probe to word 1 (SEQ ID NO:2 TATGTTGAGG/3Cy3Sp/, Integrated DNA Technologies, Newark, N.J.), and loaded on the solid support.

KG-1 cells (macrophages, ATCC) and Jurkat cells (T-cells, ATCC) were stained with NucBlue LiveReady Probes Hoechst 3342 (Thermo Fisher Scientific, Waltham, Mass.) and CellTrace CFSE, cell proliferation kit (Thermo Fisher Scientific, Waltham, Mass.), respectively, according to the manufacturer's protocols. The cells were harvested, washed with PBS (Thermo Fisher Scientific, Waltham, Mass.) and resuspended at a concentration of ˜2×10⁶ cells/mL and 3×10⁶ cells/mL and about 200,000 and 300,000 cells were successively loaded in one of the 16 channels of a solid support by flowing ˜100 μl over the solid support. The solid support was centrifuged for 5 minutes at 300 g for each loading.

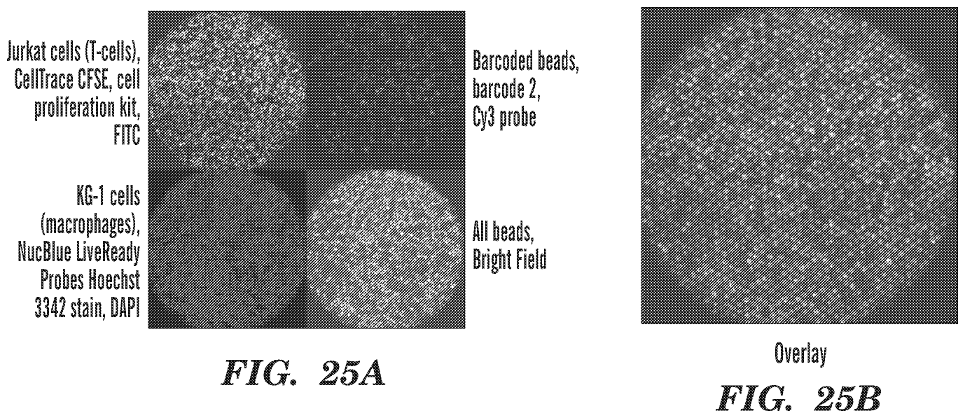

FIG. 25A shows the solid support imaged under a fluorescent upright microscope in bright field and in the GFP, RFP and DAPI fluorescent channels. All beads were detected and mapped in bright field (lower right corner of FIG. 25A). Only oligonucleotide-conjugated beads with word 1 were detected in the RFP channel (upper right corner of FIG. 25A). Oligonucleotide-conjugated beads with word 2 were not detected in the RFP channel (compare upper right corner of FIG. 25A with lower right corner of FIG. 25B). KG-1 cells were detected in the DAPI channel (lower left corner of FIG. 25A) and Jurkat cells emitted fluorescent signal in the GFP channel (upper right corner of FIG. 25A). FIG. 25B is a merged overlay of the four channels.

### Example 8—Fluorescent Signal Detection in Fiber Optic Solid Supports

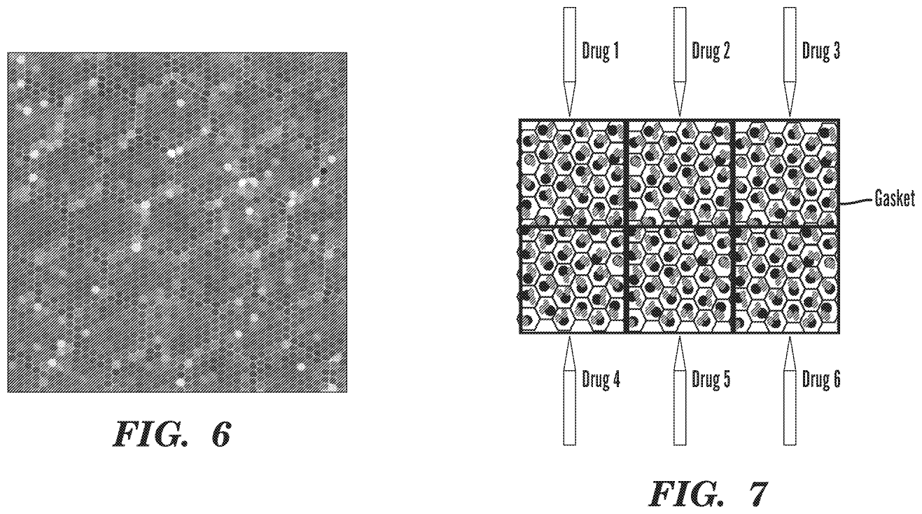

A solid support loaded with FluoSpheres Polystyrene Microspheres, 15 μm, blue fluorescent (365/415) (Thermo Fisher Scientific, Waltham, Mass.) was imaged from the bottom of the plate on an inverted fluorescent microscope (Zeiss microscopy, Jena, Germany). From the bottom, the light illuminating from the source is being captured by the core bar and the total internal reflection takes effect, causing the entire core of the fiber to illuminate leading to a bright homogeneous fluorescent signal (FIG. 6).

### Example 9—Cell Lysis in a Solid Support

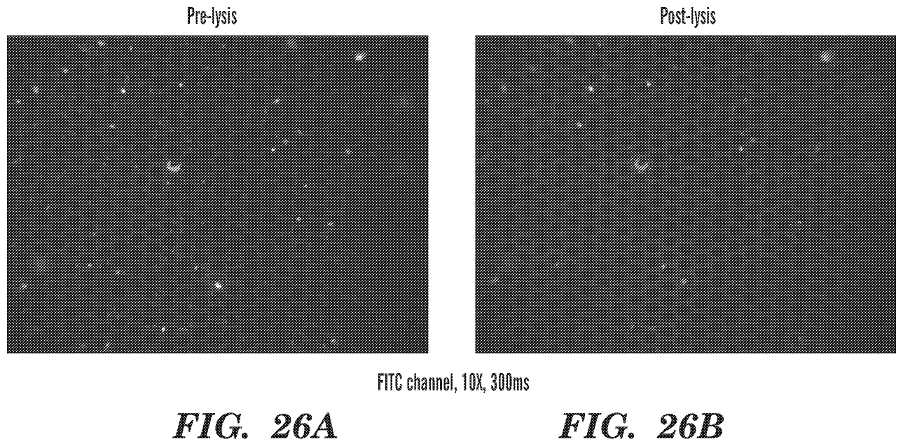

RNA from Jurkat cells (T-cells, ATCC) was stained with SYTO RNASelect Green Fluorescent stain (Thermo Fisher Scientific, Waltham, Mass.) according to the manufacturer's protocol, harvested, washed with PBS (Thermo Fisher Scientific, Waltham, Mass.) and resuspended at a concentration of ˜1×10⁶ cells/mL. About 100,000 cells were loaded in one of the 16 channels of a solid support by flowing ˜100 μl over the solid support. The solid support was centrifuged for 5 minutes at 300 g and imaged under a fluorescent upright microscope in the FITC channel (FIG. 26A). Cells were lysed by flowing 2×TCL buffer (Qiagen, Hilden, Germany) onto the solid support and incubating the plate for 5 minutes at room temperature. The solid support was imaged again after the lysis to confirm cell lysis and release of RNA (FIG. 26B).

### Example 10—Retrieval of Barcoded Material

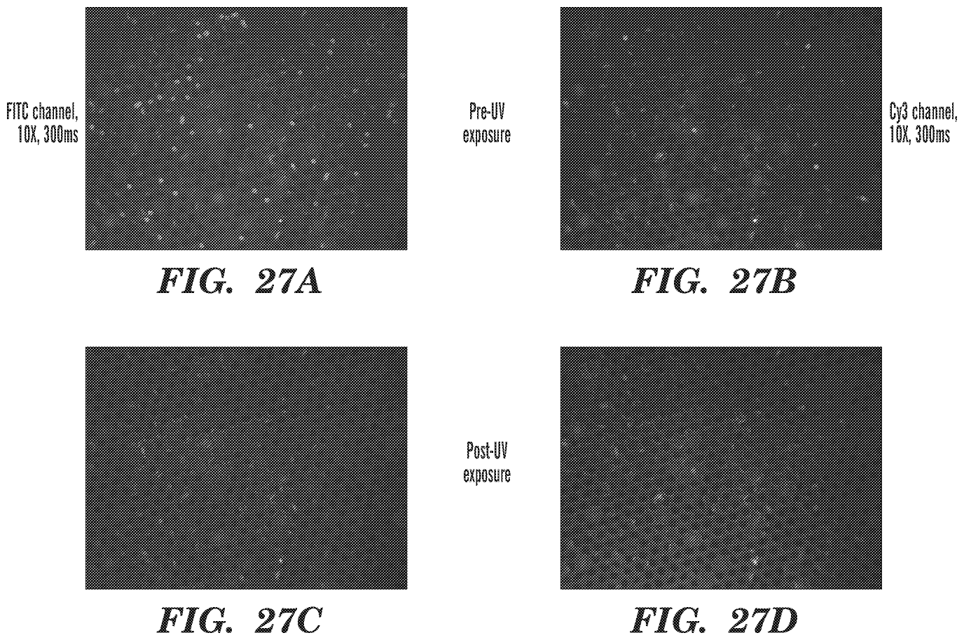

A mixture of 16 μm oligonucleotide-conjugated beads coated with photo-cleavable barcodes comprised of either word 1 and word 2 (SEQ ID NO:11 /5PCBio/ACACTCTTTCCCTACACGACGCTCTTCCGATCTAAATGTCCCGGGGTT ATAGANNNNNNNNNNTTTTTTTTTTTTTTTTTTTTVN, Integrated DNA Technologies, Newark, N.J.) or word 3 and word 4 (SEQ ID NO:12 /5PCBio/ACACTCTTTCCCTACACGACGCTCTTCCGATCTCCTCAACATATGTCC GAAAGNNNNNNNNNNTTTTTTTTTTTTTTTTTTTTVN, Integrated DNA Technologies, Newark, N.J.). The oligonucleotide-conjugated beads were incubated with a mixture of Cy3 fluorescently labelled complementary probes to word 1 (SEQ ID NO:2 TATGTTGAGG/3Cy3Sp/, Integrated DNA Technologies, Newark, N.J.) and FITC fluorescently labelled complementary probes to word 3 (SEQ ID NO:10 CGGGACATTT/3AlexF488N/, Integrated DNA Technologies, Newark, N.J.) and loaded on the solid support. This resulted in a mixture of decoded beads resulting in signal in either the FITC for the word 3 barcode (FIG. 27A) or Cy3 channel for the word 1 barcode (FIG. 27B). Oligonucleotide-conjugated beads were exposed to UV light for 5 minutes resulting in the release of the barcoded oligonucleotides from the beads. FIGS. 27 C-D show the loss of fluorescence from the beads after UV treatment.

The released oligonucleotides can be collected from the solid support. Alternatively, barcoded oligonucleotides may be attached to the beads via other reversible linkers such as a linker containing Uracils used in conjunction of the Uracil-Specific-Excision Reagent enzyme (USER, New England Biolabs, Ipswich, Mass.) for the release of such barcoded oligonucleotides.

### Example 11—Targeted SCRB-Seq

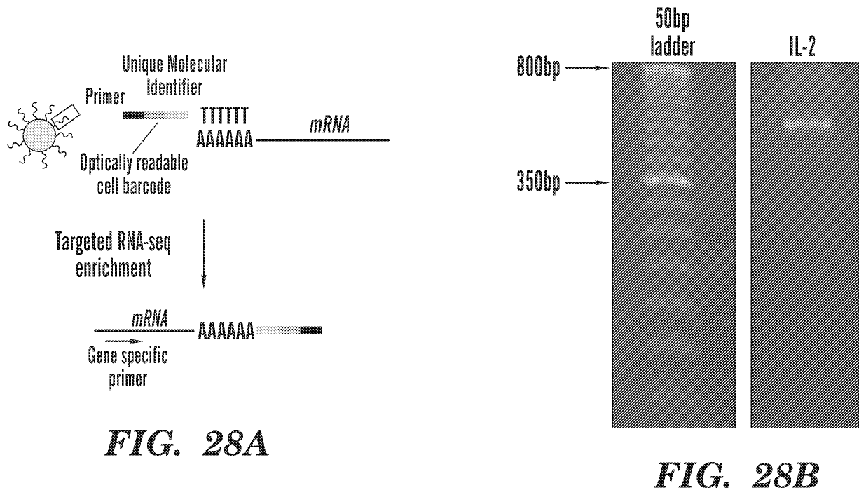

A schematic description of targeted Single Cell RNA Barcoding and Sequencing (“SCRB-seq”) is shown in FIG. 28A. In this method, the barcode anneals to the poly A tail at the 3′ end of all mRNAs. All mRNAs are converted in barcoded cDNA by template switching and amplified as described in the SCRB-seq approach (see Soumillon et al., “Characterization of Directed Differentiation by High-Throughput Single-Cell RNA-Seq,” Biokciv, 003236 (2014), which is hereby incorporated by reference in its entirety). A fraction of the barcoded cDNA is then amplified by PCR using a gene specific primer designed on the 5′ end of the mRNA and a universal primer present on the 3′ end of the barcoded cDNA. The gene specific primer carries an additional sequencing primer sequence on its 5′ end to append the gene specific enriched amplicon with template suitable for sequencing. The gene specific amplicon is then subjected to a second round of amplification by PCR using sequencing compatible universal primers that may carry a pool or experiment barcode different from the well barcode. The targeted genes may complement the results from the functional assay by measuring for example the transcripts specific to secreted proteins.

In the example shown in FIG. 28B, total RNA extracted from bulk Jurkat cells was converted in barcoded cDNA according to the SCRB-seq protocol (Soumillon et al., ibid). 1 ng of the resulting barcoded cDNA was used as input material to specifically amplify the IL-2 gene.

The sequence of Interleukin-2 (NM_000586), SEQ ID NO:13 is as follows:

CTATCACCTAAGTGTGGGCTAATGTAACAAAGAGGGATTTCACCTACATC
CATTCAGTCAGTCTTTGGGGGTTTAAAGAAATTCCAAAGAGTCATCAGAA
GAGGAAAAATGAAGGTAATGTTTTTTCAGACAGGTAAAGTCTTTGAAAAT
ATGTGTAATATGTAAAACATTTTGACACCCCCATAATATTTTTCCAGAAT
TAACAGTATAAATTGCATCTCTTGTTCAAGAGTTCCCTATCACTCTCTTT
AATCACTACTCACAGTAACCTCAACTCCTGCCACAATGTACAGGATGCAA
CTCCTGTCTTGCATTGCACTAAGTCTTGCACTTGTCACAAACAGTGCACC
TACTTCAAGTTCTACAAAGAAAACACAGCTACAACTGGAGCATTTACTGC
TGGATTTACAGATGATTTTGAATGGAATTAATAATTACAAGAATCCCAAA
CTCACCAGGATGCTCACATTTAAGTTTTACATGCCCAAGAAGGCCACAGA
ACTGAAACATCTTCAGTGTCTAGAAGAAGAACTCAAACCTCTGGAGGAAG
TGCTAAATTTAGCTCAAAGCAAAAACTTTCACTTAAGACCCAGGGACTTA
ATCAGCAATATCAACGTAATAGTTCTGGAACTAAAGGGATCTGAAACAAC
ATTCATGTGTGAATATGCTGATGAGACAGCAACCATTGTAGAATTTCTGA
ACAGATGGATTACCTTTTGTCAAAGCATCATCTCAACACTGACTTGATAA
TTAAGTGCTTCCCACTTAAAACATATCAGGCCTTCTATTTATTTAAATAT
TTAAATTTTATATTTATTGTTGAATGTATGGTTTGCTACCTATTGTAACT
ATTATTCTTAATCTTAAAACTATAAATATGGATCTTTTATGATTCTTTTT
GTAAGCCCTAGGGGCTCTAAAATGGTTTCACTTATTTATCCCAAAATATT
TATTATTATGTTGAATGTTAAATATAGTATCTATGTAGATTGGTTAGTAA
AACTATTTAATAAATTTGATAAATATAAA

The IL-2 gene specific primers was appended with Illumina read 2 sequencing primer (SEQ ID NO:14 GTCTCGTGGGCTCGGAGATGTGTATAAGAGACAGCCACAGAACTGAAACATC TTCAGT) and used in conjunction with a truncated version of Illumina read 1 sequencing primer (SEQ ID NO:15 CCCTACACGACGCTCTTCCGATCT, Integrated DNA Technologies, Newark, N.J.). IL-2 transcript specific barcoded cDNA was enriched by PCR (25 cycles) with these two primers using the Q5 master mix (New England Biolabs, Ipswich, Mass.) according to the manufacturer's instructions. PCR products were purified using 1× volume ratio of Ampure beads (Beckman Coulter, Brea, Calif.) according to the manufacturer's protocol. Purified amplicons were then subjected to a second round of amplification by PCR (25 cycles) using modified Illumina P5 sequencing primers (SEQ ID NO:16 AATGATACGGCGACCACCGAGATCT, Integrated DNA Technologies, Newark, N.J.) and Nextera N708 barcoded primer with the Q5 master mix (New England Biolabs, Ipswich, Mass.) as described by the manufacturer. Sequencing ready amplicons were purified using 1× volume ratio of Ampure beads (Beckman Coulter, Brea, Calif.) according to the manufacturer protocol and expected size was confirmed by gel electrophoresis (FIG. 28B) using E-gel EX 2% agarose gel (Thermo Fisher Scientific, Waltham, Mass.) and 50 bp ladder (Thermo Fisher Scientific, Waltham, Mass.). Several target may be enriched simultaneously by multiplex PCR. Resulting sequencing libraries may be sequenced on any Illumina instrument. When used in conjunction with the solid support system, the barcode reveals the exact position on the solid support where the sequence originated.

### Example 12—TCR-Seq and SCRB-Seq

A schematic view of T-cell receptor sequencing (“TCR-seq”) library preparation is shown in FIGS. 10A-C. In this example, total RNA extracted from a mouse lymph node and lungs was converted into barcoded cDNA as described in the SCRB-seq protocol in Soumillon et al., “Characterization of Directed Differentiation by High-Throughput Single-Cell RNA-Seq,” BioRxiv, 003236 (2014), which is hereby incorporated by reference in its entirety. TCR specific primers designed in the 5′ end of the α and β chain TCR messenger RNAs such as the ones described in Imura et al., “Role of TRPV3 in Immune Response to Development of Dermatitis,” J. Inflammation 5:17 (2009), hereby incorporated by reference in its entirety, for mouse, or in Han et al., “Linking T-Cell Receptor Sequence to Functional Phenotype at the Single Cell Level”, Nat. Biotech. 32:684-692 (2014), hereby incorporated by reference in its entirety, for humans, were used to enrich and amplify TCR sequences using the Q5 amplification master mix (New England Biolabs, Ipswich, Mass., 10 cycles) according to the manufacturer's protocol. PCR products were purified using 1.8× volume ratio of Ampure beads (Beckman Coulter, Brea, Calif.) according to the manufacturer's protocol. Purified amplicons were then subjected to a second round of amplification by PCR (10 cycles) using modified Illumina Nextera sequencing primers (SEQ ID NO:17 TCGCCTTAGTCTCGTGGGCTCGGAGATGTG, Integrated DNA Technologies, Newark, N.J.) to add a pool index and a truncated version of Illumina read 1 sequencing primer (SEQ ID NO:18 CCCTACACGACGCTCTTCCGATCT, Integrated DNA Technologies, Newark, N.J.) with the Q5 master mix (New England Biolabs, Ipswich, Mass.) as described by the manufacturer.

Resulting amplicons were purified using 1.8× volume ratio of Ampure beads (Beckman Coulter, Brea, Calif.) according to the manufacturer protocol, migrated on a 2% E-gel EX (Thermo Fisher Scientific, Waltham, Mass.) and material comprised between 850 bp and 1 kb was extracted using a MinElute Gel Extraction column (Qiagen, Hilden, Germany). Purified amplicons were phosphorylated at the 5′ end using T4 PNK (New England Biolabs, Ipswich, Mass.) according to the manufacturer protocol for subsequent ligation. The resulting product was purified using 1.8× volume ratio of Ampure beads (Beckman Coulter, Brea, Calif.) and quantified using the Qubit dsDNA high sensitivity assay (Thermo Fisher Scientific, Waltham, Mass.). 10 ng of phosphorylated amplicons were circularized using T4 ligase (Thermo Fisher Scientific, Waltham, Mass.) and overnight incubation. Circularized product was separated from non-circularized product by migration on a 2% E-gel EX (Thermo Fisher Scientific, Waltham, Mass.) and extracted using a MinElute Gel Extraction column (Qiagen, Hilden, Germany). The variable region of the TCR was enriched from the circularized TCR by nested PCR using sense and antisense primers designed on the constant region (SEQ ID NO:19 ACCATCCTCTATGAGATCCT, Integrated DNA Technologies, Newark, N.J. and SEQ ID NO:20 GGGTGGAGTCACATTTCTCAGATCC, Integrated DNA Technologies, Newark, N.J.) in the first PCR and a second set of primers designed on the constant region appended with Illumina specific sequencing primers (SEQ ID NO:21 CAAGCAGAAGACGGCATACGAGATACCATCCTCTATGAGATCCT, Integrated DNA Technologies, Newark, N.J. and SEQ ID NO:22 AATGATACGGCGACCACCGAGATCTGGGTGGAGTCACATTTCTCAGATCC, Integrated DNA Technologies, Newark, N.J.). PCR products were migrated on a 2% E-gel EX (Thermo Fisher Scientific, Waltham, Mass.) and ˜800 bp sequencing ready library was extracted using a MinElute Gel Extraction column (Qiagen, Hilden, Germany).

Sequencing was carried on an Illumina MiSeq system as a 2×300 cycles with a custom index set-up as an 8 bp Illumina index followed by a 52 random mers (N₅₂). The nucleotide sequence length distribution of the CDR3 region is shown FIG. 10C (“Flexomics”). The productive frequency of the J genes is shown in FIG. 10D, and the productive frequency of the V genes is shown in FIG. 10E. The performance of this method of TCR-seq is similar to the Adaptive Biotechnologies approach (FIG. 10F-H). When used in conjunction with the solid support system, the barcode reveals the exact position on the picowell plate where the sequence originated.

Although preferred embodiments have been depicted and described in detail herein, it will be apparent to those skilled in the relevant art that various modifications, additions, substitutions, and the like can be made without departing from the spirit of the application and these are therefore considered to be within the scope of the application as defined in the claims which follow.

What is claimed is:

1. An optically readable oligonucleotide probe comprising a plurality of nucleotide sequences, wherein each sequence of the plurality of nucleotide sequences comprises at least 3 distinct fluorescent labels comprising up to 7 unique fluorescent combinations to form a fluorescent word.

2. The probe of claim 1, wherein each of the plurality of nucleotide sequences comprises 6-20 nucleotides.

3. The probe of claim 1 or claim 2, wherein the fluorescent labels comprise fluorophores attached to one or more nucleotides.

4. The probe of any one of claims 1-3, wherein the fluorescent labels are positioned on internal positions of the plurality of nucleotide sequences.

5. The probe of any one of claims 1-4, wherein each of the fluorescent labels comprises any one of Alexa405, Alexa488, Alexa456, Alexa605, FAM, HEX, Cy3, Fluorescein-12-GTP, Cyanin-5-CTP, Cyanin-3-CTP, Cyanin-3-UTP, Fluorescein-12-ATP, Texas red-dATP, Cyanine-3-dCTP, cyanine-5-dGTP, or Fluorescein-12-dUTP.

6. The probe of any one of claims 1-5, wherein each sequence of the plurality of nucleotide sequences comprises at least 4 distinct fluorescent labels comprising up to 15 unique fluorescent combinations to form the fluorescent word.

7. The probe of any one of claims 1-5, wherein each sequence of the plurality of nucleotide sequences comprises at least 5 distinct fluorescent labels comprising up to 31 unique fluorescent combinations to form the fluorescent word.

8. The probe of any one of claims 1-5, wherein each sequence of the plurality of nucleotide sequences comprises at least 6 distinct fluorescent labels comprising up to 63 unique fluorescent combinations to form the fluorescent word.

9. The probe of any one of claims 1-5, wherein each sequence of the plurality of nucleotide sequences comprises at least 7 distinct fluorescent labels comprising up to 127 unique fluorescent combinations to form the fluorescent word.

10. The probe of any one of claims 1-9, wherein the probe comprises a single subunit DNA-dependent RNA polymerase.

11. The probe of claim 10, wherein the polymerase is any one of T7 polymerase, SP6 polymerase or T3 polymerase.

12. An oligonucleotide complex comprising:
a 5′ primer sequence;
an optically readable well-identifying barcode comprising at least two words, wherein each word comprises a unique first plurality of nucleotide sequences, wherein the first plurality of nucleotide sequences are complementary to a second plurality of nucleotide sequences in an optically readable oligonucleotide probe, wherein each nucleotide sequence of the second plurality of nucleotide sequences comprises at least 3 distinct fluorescent labels comprising up to 7 unique fluorescent combinations to form each of the fluorescent words;
a unique molecular identifier; and
a 3′ capture sequence.

13. The oligonucleotide complex of claim 12, wherein each of the words comprises 6-20 nucleotides.

14. The oligonucleotide complex of claim 12, wherein the second plurality of nucleotide sequences comprises at least 4 distinct fluorescent labels comprising up to 15 unique fluorescent combinations to form each of the fluorescent words.

15. The oligonucleotide complex of claim 12, wherein the second plurality of nucleotide sequences each comprises at least 5 distinct fluorescent labels comprising up to 31 unique fluorescent combinations to form each of the fluorescent words.

16. The oligonucleotide complex of claim 12, wherein the second plurality of nucleotide sequences each comprise at least 6 distinct fluorescent labels comprising up to 63 unique fluorescent combinations to form each of the fluorescent words.

17. The oligonucleotide complex of claim 12, wherein each sequence of the second plurality of nucleotide sequences comprises at least 7 distinct fluorescent labels comprising up to 127 unique fluorescent combinations to form each of the fluorescent words.

18. An oligonucleotide-conjugated bead comprising:
a bead and
a plurality of oligonucleotide complexes conjugated to the bead, wherein each oligonucleotide complex comprises:
a 5′ primer sequence;
an optically readable well-identifying barcode comprising at least two words, wherein each word comprises a unique first plurality of nucleotide sequences, wherein the first plurality of nucleotides are complementary to a second plurality of nucleotide sequences in an optically readable oligonucleotide probe, wherein each nucleotide sequence of the second plurality of nucleotide sequences comprises at least 3 distinct fluorescent labels comprising up to 7 unique fluorescent combinations to form each of the fluorescent words;
a plurality of unique molecular identifiers; and
a plurality of capture sequences.

19. The oligonucleotide-conjugated bead of claim 18, wherein each of the words comprises 6-20 nucleotides.

20. The oligonucleotide-conjugated bead of claim 18, wherein the second plurality of nucleotide sequences each comprise at least 4 distinct fluorescent labels comprising up to 15 unique fluorescent combinations to form each of the fluorescent words.

21. The oligonucleotide-conjugated bead of claim 18, wherein the second plurality of nucleotide sequences each comprise at least 5 distinct fluorescent labels comprising up to 31 unique fluorescent combinations to form each of the fluorescent words.

22. The oligonucleotide-conjugated bead of claim 18, wherein the second plurality of nucleotide sequences each comprise at least 6 distinct fluorescent labels comprising up to 63 unique fluorescent combinations to form each of the fluorescent words.

23. The oligonucleotide-conjugated bead of claim 18, wherein the second plurality of nucleotide sequences each comprise at least 7 distinct fluorescent labels comprising up to 127 unique fluorescent combinations to form each of the fluorescent words.

24. The oligonucleotide-conjugated bead of claim 18, wherein the plurality of capture sequences comprise more than one sequence per bead.

25. The oligonucleotide-conjugated bead of claim 24, wherein the plurality of capture sequences comprise one or more of oligo-dT sequences, gene specific primer sequences, and PCR primers.

26. The oligonucleotide-conjugated bead of claim 18, wherein the plurality of capture sequences comprise one or more of an oligo-dT sequence, a TCR sequence, a BCR sequence, a GATA3 sequence, a TBET sequence, FOXP3 sequence, RORC sequence, RUNX1 sequence, RUNX3 sequence, BCL6 sequence, IL2 sequence, IL10 sequence, IL12A sequence, IL13 sequence, IL17A sequence, IFNG sequence, TNFA sequence, TGFB sequence, PRF1 sequence, and a GZMB sequence.

27. A solid support detection system comprising:
a solid support comprising a plurality of wells and
a plurality of oligonucleotide-conjugated beads according to claim 18, wherein each of the plurality of oligonucleotide-conjugated beads comprises a unique optically readable well-identifying barcode, and wherein a plurality of the wells comprises one of the oligonucleotide-conjugated beads.

28. The solid support detection system of claim 27 further comprising:
a map identifying a well location on the solid support for each oligonucleotide-conjugated bead.

29. The solid support detection system of claim 27 further comprising:
a cell in the plurality of wells comprising one of the oligonucleotide-conjugated beads.

30. The solid support detection system of claim 29, wherein the cell is one or more of a T-cell or a B-cell.

31. The solid support detection system of claim 29 further comprising:
an interacting molecular component in the plurality of wells comprising one of the oligonucleotide beads.

32. The solid support detection system of claim 31, wherein the interacting molecular component is an Antigen Presenting Cell.

33. The solid support detection system of claim 31, wherein the interacting molecular component is an antigen-coated bead.

34. The solid support detection system of claim 29 further comprising:
a detectable product from a functional assay for a molecule of interest in the plurality of wells comprising one of the oligonucleotide beads.

35. The solid support detection system of claim 29 further comprising:
a cell-lysing agent in the plurality of wells comprising one of the oligonucleotide beads.

36. The solid support detection system of claim 29 further comprising:
a gasket, wherein the gasket divides the plurality of wells into separated areas.

37. The solid support detection system of claim 36, wherein the gasket divides the plurality of wells into 2-20 separated areas.

38. The solid support detection system of claim 27, wherein the plurality of wells comprises more than 100,000 wells.

39. A method of preparing a solid support detection system for analyzing molecular interactions, said method comprising:
providing a solid support comprising a plurality of wells;
depositing in a plurality of wells a unique, oligonucleotide-conjugated bead comprising:
oligonucleotides conjugated to the bead, wherein said oligonucleotides comprise:
a 5′ primer sequence;
a well-identifying barcode sequence comprising six words, wherein each word comprises a unique first plurality of nucleotide sequences complementary to a probe;
a unique molecular identifier; and
a 3′ capture sequence;
applying a series of flows, wherein each flow comprises a set of optically readable oligonucleotide probes, wherein each probe comprises a second plurality of nucleotide sequences complementary to the first plurality of nucleotide sequences, wherein each sequence of the second plurality of nucleotide sequences comprises at least 3 distinct fluorescent labels comprising up to 7 unique fluorescent combinations to form the words.

40. The method of claim 39, wherein each of the words comprises 6-20 nucleotides.

41. The method of claim 39 or claim 40, wherein the second plurality of nucleotide sequences each comprises at least 4 distinct fluorescent labels comprising up to 15 unique fluorescent combinations to form each of the fluorescent words.

42. The method of claim 39 or claim 40, wherein the second plurality of nucleotide sequences each comprises at least 5 distinct fluorescent labels comprising up to 31 unique fluorescent combinations to form each of the fluorescent words.

43. The method of claim 39 or claim 40, wherein the second plurality of nucleotide sequences each comprises at least 6 distinct fluorescent labels comprising up to 63 unique fluorescent combinations to form each of the fluorescent words.

44. The method of claim 39 or claim 40, wherein the second plurality of nucleotide sequences each comprises at least 7 distinct fluorescent labels comprising up to 127 unique fluorescent combinations to form each of the fluorescent words.

45. The method of claim 39 further comprising:
imaging the solid support to identify wells containing beads with one of the fluorescent words.

46. The method of claim 39, wherein said imaging is carried out after each flow in the series of flows.

47. The method of claim 39, wherein the method comprises at least 2 flows in the series, wherein each flow comprises a set of the optically readable oligonucleotide probes.

48. The method of claim 46 further comprising:
eliminating fluorescence between each flow.

49. The method of claim 39 further comprising:
mapping the location on the solid support, by well, of each oligonucleotide-conjugated bead.

50. The method of claim 39, wherein a plurality of the wells further comprise a cell.

51. The method of claim 50, wherein the cell is one or more of a T-cell or a B-cell.

52. The method of claim 50, wherein a plurality of the wells further comprises an interacting molecular component.

53. The method of claim 52, wherein a plurality of the wells further comprises an interacting molecular component and the interacting molecular component is an Antigen Presenting Cell.

54. The method of claim 52, wherein a plurality of the wells further comprises an interacting molecular component and the interacting molecular component is an antigen-coated bead.

55. The method of claim 50 further comprising:
applying a gasket to the solid support to divide the plurality of wells into separated areas.

56. The method of claim 55, wherein the gasket is applied to divide the plurality of wells into 2-20 separated areas.

57. The method of claim 56 further comprising:
applying a chemical compound to the separated areas.

58. The method of claim 39, wherein the plurality of wells comprises more than 100,000 wells.

59. The method of any one of claims 50-58 further comprising:
detecting a product from a functional assay for a molecule of interest.

60. The method of any one of claims 50-59 further comprising:
lysing the cells.

61. The method of any one of claims 50-60 further comprising:
detecting nucleotide sequences from the molecular components.

62. The method of claim 61, wherein the nucleotide sequences from the molecular components comprise a well-identifying barcode and a sequence derived from an RNA molecule or a DNA molecule.

63. The method of claim 62, wherein the nucleotide sequences derived from the RNA molecule or the DNA molecule comprise any one or more of a TCR sequence, a BCR sequence, a GATA3 sequence, a TBET sequence, FOXP3 sequence, RORC sequence, RUNX1 sequence, RUNX3 sequence, BCL6 sequence, IL2 sequence, IL10 sequence, IL12A sequence, IL13 sequence, IL17A sequence, IFNG sequence, TNFA sequence, TGFB sequence, PRF1 sequence, and a GZMB sequence.

64. The method of any one of claims 61-63 further comprising:
identifying the well location of the nucleotide sequences using the well-identifying barcode and
re-enriching the nucleotide sequences from target wells for downstream applications using the well-identifying barcode as a primer sequence.

65. A high-throughput method of analyzing molecular interactions, said method comprising:
providing the solid support detection system according to claim 27;
contacting the solid support with molecular components to be analyzed, wherein the molecular components are deposited in the plurality of wells;
detecting phenotypic interactions between said molecular components;
capturing RNA or DNA derived from the molecular components;
transcribing captured RNA to DNA;
enriching the DNA;
sequencing the DNA; and
matching sequences from the sequenced DNA to barcode sequences to identify the well location and, therefore, molecular interactions of interest among the molecular components.
